(19) United States
(12) Patent Application Publication
(10) Pub. No.: US 2026/0248525 A1
(43) Pub. Date: Aug. 27, 2026

(54) SURGICAL INSTRUMENT WITH SWIVELING END EFFECTOR AND METHOD OF USE THEREOF

(71) Applicant: TAG Dream Medical LTD, Yokneam Ilit (IL)

(72) Inventors: Dan MICHAEL, Moshav Beit Shearim (IL); Dan NABEL, Kiryat-Tivon (IL); Yuval PERETS, Moshav Beit Shearim (IL); Yaron TAL, Tel-Mond (IL)

(73) Assignee: TAG Dream Medical LTD, Yokneam Ilit (IL)

(21) Appl. No.: 19/162,175

(22) PCT Filed: Mar. 6, 2024
(86) PCT No.: PCT/IL2024/050242

§ 371 Date: Sep. 4, 2025

Related U.S. Application Data
(60) Provisional application No. 63/450,367, filed on Mar. 6, 2023.

(51) Int. Cl. A61B 17/295; A61B 17/00; A61B 17/29

(52) U.S. Cl. CPC A61B 17/295; A61B 2017/00473; A61B 2017/2927

(57) ABSTRACT

A surgical instrument comprising a swiveling end effector at a distal portion of the surgical instrument, the swiveling end effector having first and second jaws, the first jaw movable between open and closed positions relative to the second jaw, wherein the first jaw is actuatable by application of a force at a proximal portion of the surgical instrument, and wherein the surgical instrument is configured to deliver a torque at the first jaw when moving between the open and closed positions; and a swiveling actuator coupled to the swiveling end effector and operative to swivel the swiveling end effector; wherein the surgical instrument is operable to deliver a substantially same torque at the first jaw when moving between the open and closed positions, regardless of a swivel position of the swiveling end effector relative to the swivel axis.

### RELATED APPLICATION/S

This application claims the benefit of priority of U.S. Provisional Patent Application No. 63/450,367, filed on Mar. 6, 2023, the contents of which are incorporated herein by reference in their entirety.

### FIELD AND BACKGROUND OF THE INVENTION

The present disclosure is directed to a surgical instrument having a swiveling end effector and methods of use thereof. More specifically, the disclosure is directed to a surgical instrument comprising a swiveling end effector having a first jaw and a second jaw, the first jaw movable between an open position and a closed position at least partially abutting the second jaw, the surgical instrument operable to deliver a predetermined torque for closing the first jaw that is independent of a yaw swivel angle. Arthroscopic minimally invasive surgery with its small incision, less trauma, less scarring, faster recovery and fewer complications, relative to non-arthroscopic surgery in which a body cavity may be exposed, has gradually become a routine procedure in diagnosis and treatment in orthopedics. Current procedures typically require a myriad of angled end effectors to carry out the procedures necessary, increasing the time to perform these procedures and increasing their complexity, as well as increasing the possibility for trauma, injury (e.g., to cartilage), and infection.

Movement of the end effector through multiple angles relative to the instrument shaft is conventionally referred to as “articulation.” Articulation is typically accomplished by a pivot (or articulation) joint being placed in the elongate shaft proximal to the end effector. This allows the clinician to articulate the end effector remotely to either side for better and easier tissue manipulation and orientation. An articulating end effector permits the clinician to more easily engage tissue in some instances, such as behind an organ. In addition, articulated positioning advantageously allows an endoscope to be positioned behind the end effector without being blocked by the elongate shaft.

In addition to articulation achieved by a single pivot point achieved typically by rotating the shaft the change in the yaw angle may be advantageous. Likewise, an increased degree of freedom of the end effector in terms of the degree of yaw articulation will increase the flexibility of the device for the clinician. That flexibility can only be achieved if the load, or the force exerted by the jaws of the instrument remains consistent at any angle.

Furthermore, in performing many surgical procedures, it may be desirable to affect a desired amount of end effector articulation and rotation by using only one hand. For example, many vascular operations require precise control of the end effector. In such applications, it would be desirable to be able to have a surgical instrument that employs a single control mechanism for selectively articulating and rotating (i.e., swiveling) the end effector that can be easily actuated by using the same hand that is supporting the handle portion of the instrument.

### SUMMARY

In an exemplary implementation, provided herein is a surgical instrument comprising a swiveling end effector having a first jaw and a second jaw, the first jaw movable between an open position and a closed position at least partially abutting the second jaw, the surgical instrument operable to deliver a predetermined torque for closing the first jaw that is independent of a yaw swivel angle.

In another exemplary implementation, provided herein is a surgical instrument comprising: a swiveling end effector operable to operate at a constant load at any yaw swivel angle; an elongated cylindrical shaft defining a longitudinal axis, having a distal end operably coupled to said swiveling end effector, and a proximal end operably coupled to a stationary handle; the stationary handle; an articulating handle, hingedly coupled to the stationary handle and operably coupled to a first jaw in the swiveling end effector, configured to operate the swiveling end effector; and a swiveling member, optionally including a ratchet mechanism, operably coupled to a second jaw in the swiveling end effector, operable to swivel the swiveling end effector to a yaw swivel angle of ±120 degrees from the longitudinal axis, wherein the swiveling end effector is operable to transition between a closed position whereby the second jaw may be partially accommodated within the first jaw, and an open position whereby the distal end of the second jaw may be at a distance from the first jaw.

According to an aspect of some embodiments of the present invention there is provided a surgical instrument comprising a longitudinal axis and a swiveling end effector at a distal portion of the surgical instrument, the swiveling end effector having a first jaw and a second jaw, the first jaw movable between an open position and a closed position relative to the second jaw, wherein the first jaw is actuatable by application of a force at a proximal portion of the surgical instrument and wherein the surgical instrument is configured to deliver a torque at the first jaw when moving between the open position and the closed position;
wherein the swiveling end effector is configured to swivel about a swivel axis perpendicular to the longitudinal axis;
wherein the surgical instrument is operable to deliver a substantially same torque at the first jaw when moving between the open position and the closed position, regardless of a swivel position of the swiveling end effector relative to the swivel axis.

According to some embodiments, when in the closed position the first jaw at least partially abuts the second jaw.

According to some embodiments, the surgical instrument includes a shaft extending from a proximal portion of the surgical instrument to the end effector at a distal portion of the surgical instrument, the shaft having a diameter of less than 4.5 mm, and wherein the surgical instrument is configured to generate torque on the first jaw of from about 150 N·mm to about 600 N·mm on the jaws.

According to some embodiments, the swiveling end effector is one of a punch, a grasper, a suture passer, bypass scissors, and an anvil scissors.

According to some embodiments, there is provided a kit including:
the surgical instrument of claim 1; and
at least one additional swiveling end effector, wherein the swiveling end effector is replaceable with the additional swiveling end effector.

According to some embodiments, the swiveling end effector is configured to swivel about ±120 degrees relative to the longitudinal axis, wherein the surgical instrument is operable to deliver a substantially same torque at the first jaw when moving between the open position and the closed position, at any swivel position of the swiveling end effector relative to the swivel axis selected from −120 degrees to +120 degrees.

According to some embodiments, the swiveling end effector is configured to swivel ±90 degrees relative to the longitudinal axis.

According to some embodiments, the swiveling end effector is configured to swivel ±120 degrees relative to the longitudinal axis.

According to some embodiments, the end effector is configured to swivel about a swivel axis; and
wherein the surgical instrument includes an end effector actuating assembly having a joint at which the first jaw is configured to hinge, wherein the swivel axis extends through the joint.

According to some embodiments, the end effector is configured to swivel about a swivel axis; and
wherein the surgical instrument includes an end effector actuating assembly having a joint at which the first jaw is configured to hinge, wherein the joint is a ball joint.

According to some embodiments, the end effector includes a surgical tool configured to bite a meniscus in a knee.

According to some embodiments, the surgical instrument is operable to deliver sufficient torque at the first jaw to move between the open position and the closed position.

According to an aspect of some embodiments of the present invention there is provided a surgical instrument comprising a longitudinal axis and a swiveling end effector at a distal portion of the surgical instrument, the swiveling end effector having a first jaw and a second jaw, the first jaw movable between an open position and a closed position relative to the second jaw, wherein the first jaw is actuatable by application of a force at a proximal portion of the surgical instrument and wherein the surgical instrument is configured to deliver a torque at the first jaw when moving between the open position and the closed position;
wherein the swiveling end effector is configured to swivel about a swivel axis perpendicular to the longitudinal axis;
wherein the surgical instrument is operable to deliver at the first jaw sufficient torque to move the first jaw between the open position and the closed position.

According to some embodiments, the surgical instrument includes a shaft extending from the surgical instrument proximal portion to the surgical instrument distal portion, and wherein the surgical instrument is configured to generate torque on the first jaw of from about 150 N·mm to about 600 N·mm on the jaws, for a shaft diameter of less than 4.5 mm.

According to some embodiments, the swiveling end effector is a surgical tool configured to bite or cut through a meniscus, the first jaw configured to at least partly abut the second jaw when in the closed position.

According to an aspect of some embodiments of the present invention there is provided a surgical instrument comprising a longitudinal axis and a swiveling end effector at a distal portion of the surgical instrument, the swiveling end effector having a first jaw and a second jaw, the first jaw movable between an open position and a closed position relative to the second jaw, wherein the first jaw is actuatable by application of a force at a proximal portion of the surgical instrument and wherein the surgical instrument is configured to deliver a torque at the first jaw when moving between the open position and the closed position;
wherein the swiveling end effector is configured to swivel about a swivel axis perpendicular to the longitudinal axis; and
wherein the swiveling end effector is configured to swivel about the swivel axis in a clockwise direction and in a counterclockwise direction.

According to an aspect of some embodiments of the present invention there is provided an arthroscopic surgical instrument having a longitudinal axis and an end effector including a surgical tool configured to bite a meniscus in a knee, the surgical tool having a first jaw and a second jaw, the first jaw manually actuatable to move between an open position and a closed position relative to the second jaw;
wherein the end effector is configured to swivel about a swivel axis perpendicular to the longitudinal axis.

According to some embodiments, the swiveling end effector is configured to swivel to a swivel angle of ±120 degrees about the swivel axis.

According to some embodiments, the surgical instrument includes a shaft extending from a surgical instrument proximal portion to a surgical instrument distal portion, wherein the end effector is located at the surgical instrument distal portion; and
wherein the surgical instrument is configured to generate torque on the first jaw of from about 150 N·mm to about 600 N·mm on the jaws, for a shaft diameter of less than 4.5 mm.

According to some embodiments, the surgical tool has a length of 5.5 mm;
wherein the shaft has an outer diameter of 4.2 mm; and
wherein the surgical instrument is configured transmit a force applied at a proximal portion of the surgical instrument to generate torque of about 525 N-mm at a distal portion of the end effector, regardless of a swivel angle of the end effector relative to the longitudinal axis.

According to some embodiments, the swiveling end effector is configured to swivel both clockwise and counterclockwise about the swivel axis.

According to some embodiments, the swiveling end effector is removable and replaceable by an additional swiveling end effector.

According to some embodiments, the swiveling end effector is configured to swivel any amount, up to a swivel angle of 90 degrees about the swivel axis.

According to some embodiments, the surgical instrument includes a proximal portion having an actuator configured to receive a force and wherein the surgical instrument includes a distal portion including the end effector, wherein the actuator is configured to transmit a force applied thereat and to convert the transmitted force to the torque to be applied to the first jaw to move the first jaw from the open position to the closed position.

According to some embodiments, the surgical instrument includes a rotatable shaft having a distal end including a first gear, wherein the second jaw includes a proximal portion having a second gear, and wherein the first gear is configured to engage with the second gear whereby rotation of the shaft causes the first gear to turn the second gear thereby swiveling the swiveling end effector.

According to an aspect of some embodiments of the present invention there is provided a method of using a surgical instrument having a swiveling end effector including first and second jaws, the method including:
a. swiveling the end effector to a first swivel position at which the end effector is at a first selected angle relative to a longitudinal axis of the surgical instrument; and
b. applying torque in a first direction to a portion of the end effector to move the first jaw relative to the second jaw, from an open position to a closed position, wherein the amount of torque required to move the first jaw from the open position to the closed position is not dependent on a swivel position of the swiveling end effector;
wherein action (b) may be performed before action (a).

According to some embodiments, the method further includes applying torque in a second direction to the portion of the end effector to move the first jaw relative to the second jaw, from the closed position to the open position, wherein the second direction is opposite to the first direction.

According to some embodiments, the surgical instrument includes a shaft and wherein the swiveling includes rotating a swiveling actuator a selected radial distance relative to the shaft to move the end effector from a first swivel orientation to a second swivel orientation.

According to some embodiments, the applying torque and the rotating a swiveling actuator may be performed with a same hand.

According to some embodiments, the surgical instrument includes a shaft having a longitudinal axis, and wherein the swiveling includes rotating the end effector about a swiveling axis, wherein the swiveling axis extends through the shaft longitudinal axis.

According to some embodiments, the method further includes:
c. swiveling the end effector to a second swivel position at which the end effector is at a second selected angle relative to the longitudinal axis of the surgical instrument.

According to some embodiments, the surgical instrument includes proximal and distal handles, and wherein the applying torque includes moving the proximal handle relative to the distal handle.

According to some embodiments, the surgical instrument includes proximal and distal handles, and wherein the applying torque includes moving the distal handle relative to the proximal handle.

According to some embodiments, the surgical instrument includes an actuator having an axially displaceable first portion and wherein the first jaw includes a second portion configured to mate with the first portion, and wherein the applying torque includes axially displacing the first portion to apply torque to the second portion to move the first jaw relative to the second jaw.

According to some embodiments, the axially displaceable first portion includes a portion having at least a partially circular or spherical or cylindrical configuration, and wherein the second portion includes a depression or recess sized and shaped to correspond to the first portion.

According to some embodiments, the second portion includes a portion having at least a partially circular or spherical or cylindrical configuration, and wherein the axially displaceable first portion includes a depression or recess sized and shaped to correspond to the second portion.

According to some embodiments, the surgical instrument includes a shaft having a diameter of less than 4.5 mm, and wherein the applying a torque includes applying a torque of from about 150 N·mm to about 600 N·mm to the portion of the end effector.

According to an aspect of some embodiments of the present invention there is provided a method of using a surgical instrument having a longitudinal axis and a swiveling end effector including a surgical tool configured to bite a meniscus, the surgical tool having first and second jaws, wherein the surgical instrument includes a longitudinal shaft having an outer diameter of 4.2 mm, wherein the surgical instrument is configured transmit a force applied at a proximal portion of the surgical instrument to generate torque of about 525 N-mm at a distal portion of the end effector, regardless of a swivel angle of the end effector relative to the longitudinal axis, the method including:
a. swiveling the end effector to a first swivel position at which the end effector is at a first selected angle relative to a longitudinal axis of the surgical instrument;
b. applying torque in a first direction to a portion of the end effector to move the first jaw relative to the second jaw, from an open position to a closed position;
wherein action (b) may be performed before action (a); and
c. swiveling the end effector to a second swivel position at which the end effector is at a second selected angle relative to the longitudinal axis of the surgical instrument.

According to an aspect of some embodiments, there is provided a surgical instrument comprising a swiveling end effector having a first jaw and a second jaw, the first jaw movable between an open position and a closed position partially abutting the second jaw, the surgical instrument operable to deliver a predetermined torque for closing the first jaw that is independent of a yaw swivel angle.

According to some embodiments, the swiveling end effector is a punch, a grasper, a suture passer, bypass scissors, anvil scissors, or an end effector having the first jaw and the second jaw.

According to some embodiments, the surgical instrument further comprises:
a. an elongated cylindrical shaft defining a longitudinal axis, having a distal end operably coupled to the swiveling end effector, and a proximal end operably coupled to a stationary handle;
b. the stationary handle;
c. an articulating handle, hingedly coupled to the stationary handle and operably coupled to a first jaw in the swiveling end effector, configured to operate the swiveling end effector; and
d. a swiveling member, operably coupled to a second jaw in the swiveling end effector, operable to swivel the swiveling end effector to a yaw swivel angle of ±120 degrees from the longitudinal axis, wherein the second jaw partially abuts the first jaw.

According to some embodiments, the shaft comprises:
a. a sleeve, having a proximal end coupled to the stationary handle, and a distal end coupled to a ventral coupling member and a dorsal coupling member;
b. an elongated cannula nested within- and coaxial with the sleeve, having a proximal end operably coupled to the swiveling member, and a distal end having a partially cylindrical extension defining a vertical partial bevel gear at its distal end; and
c. a rod assembly, nested within the elongated cannula, the rod assembly having a proximal end operably coupled to the articulating handle, and a distal end coupled to an end effector actuating assembly.

According to some embodiments, the second jaw having a distal end and a proximal end, further comprising:
a. a partially circular extension extending proximally from the proximal end of the second jaw, the partially circular extension having a basal surface and an apical surface; and
b. a horizontal partial beveled gear, or face gear, extending apically from the apical surface of the partially circular extension, the horizontal partial beveled gear defining a coaxial aperture extending through the basal surface of the partially circular extension, wherein the horizontal partial beveled gear is configured to engage the vertical partial bevel gear at the distal end of the elongated cannula;
c. an apically flaring open portion, forming a spilt shelf defining a groove sized and configured to accommodate a portion of the first jaw, the open portion defining a proximal end, sized and configured to provide a yaw swivel freedom of about ±2.09 rad.;
d. a pair of coaxial horizontal bores defined within the apically flaring open portion transverse to the groove defined by the split shelf;
e. a dorsal hinge member, operably coupled apically to the split shelf, having a portion extending proximally from the shelf, defining an aperture therein, the aperture being coaxial with the aperture defined by the horizontal partial beveled gear coupled to apical surface of the partially circular extension.

According to some embodiments, the first jaw comprises:
a. a head portion having a distal end;
b. a lever having a front section, a mid-section, and a rear section extending proximally from the head portion, the front section and mid-section adapted sized and configured to be partially accommodated within the groove defined by the apically flaring open portion, wherein:
   i. the mid-section further defines a through hole, sized and configured to pivotally couple to the apically flaring open portion of the second jaw; and
   ii. the rear section defining a circular depression.

According to some embodiments, the end effector actuating assembly further comprises:
a. a camming element, having an L-shape with a first leg having a ball joint disposed at the distal end of the first leg, while the proximal end of the first leg is coupled to a second leg having a distal end defining a through hole, transverse to the second leg, and a proximal end, terminating in a pair of lateral extensions forming a gap there between, each lateral extension defining a pair of coaxial apertures disposed toward the proximal end of each of the pair of lateral extensions, wherein contour of the circular depression defined in the rear section of the lever extending proximally from the head portion of the second jaw is complimentary to the ball joint, and is configured to abut a portion of the ball joint;
   b. a pair of slider slabs, each having a narrow distal facet that is larger than an opposing narrow proximal facet, with a slider slab aperture defined through the slab in proximity to the basal portion of the slider slab towards the distal facet, and a tab extending from the upper narrow facet of the slab, the tab having a distal wall and a proximal wall;
c. a first hinging element, pivotally coupling the pair of slider slabs to the camming element via the through hole defined in the distal end of the second leg of the L-shape camming element; and
d. a second hinging element, pivotally coupling the second jaw and the first jaw by extending through the through hole defined in the mid-section of the lever extending proximally from the head portion of the first jaw, disposed within the groove defined by the split shelf and being coaxial with the pair of coaxial horizontal bores defined within the apically flaring open portion transverse to the groove.

According to some embodiments, the end effector actuating assembly further comprises:
a. an elongated slider having a proximal end coupled to the distal end of the rod assembly, and a distal end, the slider further having an upper surface defining a channel having a sloped portion and a horizontal portion, the upper surface configured to partially accommodate a complementary basal surface of a camming arm, wherein the distal end further includes a pair of extensions extending distally, defining an axial groove adapted sized and configured to partially accommodate a coupling slab, each of the pair of extensions further defines a coaxial horizontal bores transverse to the axial groove;
b. the coupling slab having an arcuate distal end and an arcuate proximal end, the diameter defined by the arcuate proximal end being larger than the diameter defined by the arcuate proximal end, the coupling slab defining a distal through hole and a proximal through hole;
c. the camming arm having a proximal end and a frusto-spheroid distal end, a distal portion and a proximal portion, wherein the distal portion further defines a cavity configured to accommodate the arcuate distal end of the coupling slab, the distal portion further defines a pair of apertures sized, adapted and configured coaxially with the distal through hole defined in the arcuate distal end of the coupling slab, and wherein the proximal portion further comprise a drill hole defined towards the proximal end;
d. a pair of bracket slabs, each having a narrow distal facet and an opposing narrow proximal facet, with a bracket slab aperture defined through the slab in proximity to a proximal end of the slider bracket, and a tab extending apically from the upper narrow facet of the bracket slab, the tab having a distal wall and a proximal wall;
e. a seventh hinging element, pivotally coupling the pair of slider brackets to the drill hole defined towards the proximal end of the camming arm by extending through the bracket slab aperture defined through each bracket slab in proximity to a proximal end of the bracket slab;
f. an eighth hinging element, pivotally coupling the second jaw and the first jaw by extending through the through hole defined in the mid-section of the lever extending proximally from the head portion of the first jaw, disposed within the groove defined by the split shelf and being coaxial with the pair of coaxial horizontal bores defined within the apically flaring open portion transverse to the groove;
g. a ninth hinging element, pivotally coupling the coupling slab and the camming arm, by extending through the pair of apertures defined in the distal portion of the camming arm and the coaxial distal through hole defined in the arcuate distal end of the coupling slab accommodated in the cavity defined in the distal portion of the camming element; and
h. a tenth hinging element, pivotally coupling the elongated slider and the coupling slab, by extending through the coaxial horizontal bores transverse to the axial groove defined by the pair of extensions extending distally from the elongated slider, defining the axial groove adapted sized and configured to partially accommodate the arcuate proximal end of the coupling slab, and the coaxially disposed proximal through hole.

According to some embodiments, the swiveling end effector further comprises:
a. a dorsal coupling member, coupled to- and extending distally from the distal end of the shaft, the dorsal coupling member comprising:
   i. a distal portion, defining a dorsal aperture, the dorsal aperture being coaxial with the aperture defined in the dorsal hinge member portion extending proximally from the split shelf of the second jaw; and
   ii. a proximal portion, defining a rectangular window, the rectangular window's narrow aspect sized and configured to accommodate the pair of lateral extensions of the second leg of the camming element, sandwiched between the pair of tabs flaring apically from each slider slabs;
b. a ventral coupling member, coupled to- and having a distal portion extending distally from the distal end of the shaft, the ventral coupling member comprising a distal portion defining a ventral aperture being coaxial with the coaxial aperture of the horizontal partial beveled gear, extending through the basal surface of the partially circular extension of the second jaw; and
c. a third hinging element, pivotally coupling the dorsal hinge member coupled to the split shelf of the second jaw extending proximally, and the distal portion of the dorsal coupling member via extending through the aperture defined in the distal portion of the dorsal coupling member, and the aperture defined in the dorsal hinge member: and
   d. a fourth hinging element, pivotally coupling the ventral coupling member and the basal surface of the partially circular extension of the second jaw, by extending through the ventral aperture of the ventral coupling member, and the coaxial aperture of the horizontal partial beveled gear, extending through the basal surface of the partially circular extension.

According to some embodiments, the swiveling end effector further comprises:
a. a dorsal coupling member, coupled to- and extending distally from the distal end of the shaft, the dorsal coupling member comprising:
   i. a distal portion, defining a dorsal aperture, the dorsal aperture being coaxial with the aperture defined in the dorsal hinge member portion extending proximally from the split shelf of the second jaw; and
   ii. a proximal portion, defining a rectangular window, the rectangular window's narrow aspect sized and configured to accommodate the pair tabs extending apically from the upper narrow facet of each of the bracket slab, and the proximal portion of the camming arm sandwiched between the pair of tabs;
b. a ventral coupling member, coupled to and having a distal portion extending distally from the distal end of the shaft, the ventral coupling member comprising a distal portion defining a ventral aperture being coaxial with the coaxial aperture of the horizontal partial beveled gear, extending through the basal surface of the partially circular extension of the second jaw; and
c. a third hinging element, pivotally coupling the dorsal hinge member coupled to the split shelf of the second jaw extending proximally, and the distal portion of the dorsal coupling member via extending through the aperture defined in the distal portion of the dorsal coupling member, and the aperture defined in the dorsal hinge member: and
d. a fourth hinging element, pivotally coupling the ventral coupling member and the basal surface of the partially circular extension of the second jaw, by extending through the ventral aperture of the ventral coupling member, and the coaxial aperture of the horizontal partial beveled gear, extending through the basal surface of the partially circular extension.

According to some embodiments, the stationary handle further comprises a head portion having a distal end and a proximal end, and a body portion, wherein the head portion defines:
a. a sleeve bore coaxial with the longitudinal axis of the shaft, the sleeve bore having a distal end and a proximal end;
b. a first cavity expanding from the proximal end of the sleeve bore, the first cavity adapted, sized and configured to accommodate a portion of the swiveling ratchet; and
c. a second cavity, adapted sized and configured to accommodate a portion of the articulating handle and a portion of the rod assembly.

According to some embodiments, the head portion further defines:
a. a hinge aperture configured to receive a sixth hinging element;
b. a proximal slot extending through the proximal end of the head portion, through the hinge aperture, to the second cavity; and
c. a partially circular ventral slot, flaring apically to the first cavity.

According to some embodiments, the articulating handle comprises a linking member, flaring apically from the distal end of the articulating handle, the linking member sized and configured to be accommodated in the proximal slot of the head portion of the stationary handle, further defining an apical protrusion, operable to pivotally couple to a proximal member, and wherein the linking member is hingedly coupled to the stationary handle via a sixth hinging element.

According to some embodiments, the rod assembly comprises:
a. a push-rod having a proximal end and a distal end
b. the proximal member extending proximally from the proximal end of the push rod; and
c. a camming linker, flaring apically and distally at a predetermined angle from the distal end of the push rod, the camming linker is sized to be accommodated within the gap formed by pair of lateral extensions of the camming element, and is operable to hingedly couple to the camming element via a fifth hinging element extending through the pair of coaxial apertures disposed toward the proximal end of each of the pair of lateral extensions and the camming linker sandwiched there between.

According to some embodiments, the swiveling member comprises:
a. a basal body portion defining a right depression and a left depression;
b. a flat neck portion, sized and configured to be accommodated within the partially circular ventral slot of the head portion of the stationary handle;
c. a hollow cylinder extending transverse to the neck portion, wherein the hollow cylinder is configured to couple to the proximal end of the elongated cannula; and
d. an arcuate indexing teeth portion disposed in parallel with the flat neck portion and transverse to the longitudinal axis defined by the hollow cylinder.

According to some embodiments, the first cavity further defines a radial channel configured to accommodate the arcuate indexing teeth portion, the first cavity further comprising a resilient tab, disposed transverse to-, and within, the radial channel, the resilient tab sized adapted and configured to releasably engage a tooth in the arcuate indexing teeth portion.

According to some embodiments, the hollow cylinder of the swiveling member has the same outer diameter as the shaft sleeve.

According to some embodiments, the elongated cannula further comprises a ventral opening disposed toward the proximal end of the elongated cannula.

According to some embodiments, the stationary handle further comprises a head portion having a distal end and a proximal end, and a body portion, wherein the head portion defines:
a. a sleeve bore coaxial with the longitudinal axis of the shaft, the sleeve bore having a distal end and a proximal end, the sleeve bore being adapted, sized and configured to accommodate a portion of the swiveling member; and
b. a cavity, adapted sized and configured to accommodate a biasing element and an indexing bearing.

According to some embodiments, the head portion further defines:
a. a hinge aperture configured to receive a sixth hinging element;
b. a slot extending through a basal surface of the head portion, and through the hinge aperture, to the sleeve bore; and
c. a partially circular dorsal slot, extending to the first cavity.

According to some embodiments, the rod assembly comprises:
a. a push-rod having a proximal end and a distal end; and
b. a camming linker, flaring apically and distally at a predetermined angle from the distal end of the push rod, the camming linker is sized to be accommodated within the gap formed by pair of lateral extensions of the camming element, and is operable to hingedly couple to the camming element via a fifth hinging element extending through the pair of coaxial apertures disposed toward the proximal end of each of the pair of lateral extensions and the camming linker sandwiched there between.

According to some embodiments, the articulating handle configured to be accommodated in the slot extending through a basal surface of the head portion of the stationary handle, further defining an apical protrusion, operable to pivotally couple to the proximal end of the push-rod, and wherein the apical protrusion is hingedly coupled to the stationary handle via a sixth hinging element.

According to some embodiments, the swiveling member comprises:
a. a body portion defining a right depression and a left depression;
b. a flat neck portion coupled to the body portion, defining a partially circular surface, coupled to the proximal end of the elongated cannula, the flat neck portion sized and configured to be accommodated within the partially circular dorsal slot of the head portion of the stationary handle, wherein the partially circular surface further defines an arcuate array of indexing holes or indexing depressions.

According to some embodiments, the first cavity further comprises the biasing element, operable to bias the bearing to engage one of the indexing holes or indexing depressions defined in the partially circular surface of the flat neck portion of the swiveling member.

According to some embodiments, the swiveling end effector is configured to provide a compression load of between about 29.42 newton and about 98.00 newton at any yaw swivel angle between the second jaw and the first jaw.

According to some embodiments, the shaft has an outer diameter that is no more than 4.5 mm.

According to some embodiments, the punch is an upbiter curved at an angle of between about 1° and about 15° upward at the tip.

According to some embodiments, the shaft is further swept up at a predetermined curvature.

According to some embodiments, the predetermined curvature is adapted and sized to follow a contour of the condyle, configured to maintain a lower surface of the first jaw in parallel with a tibial plateau.

According to some embodiments, the shaft comprises:
a) a sleeve, having a proximal end coupled to the stationary handle, and a flaring distal end hingedly coupled to the end effector, wherein the sleeve further defining:
   i. a ventral opening, defined toward the distal end of the sleeve, configured to accommodate a portion of an end effector actuating assembly;
   ii. a dorsal hinging plateau defined in the distal end of the sleeve; and
   iii. a ventral hinging plateau defined in the distal end of the sleeve;
b) an elongated interrupted cannula nested within- and coaxial with the sleeve, having a proximal end operably coupled to the swiveling member, and a distal end defining a horizontal spur gear at its distal end; and
c) a rod assembly, nested within the elongated cannula, the rod assembly having a proximal end operably coupled to the articulating handle, and a distal end coupled to the end effector actuating assembly.

According to some embodiments, the second jaw having a distal end and a proximal end, further comprising:
a) a partially circular extension extending proximally from the proximal end of the second jaw, the partially circular extension having a basal surface and an apical surface;
b) a horizontal partial spur gear, extending from the partially circular extension, the horizontal partial spur gear defining a coaxial aperture extending through the basal surface of the partially circular extension, wherein the horizontal partial spur gear is configured to engage the partial spur gear at the distal end of the elongated cannula;
c) an apically flaring open portion, forming a shelf defining an opening sized and configured to accommodate a portion of the first jaw, the open portion defining a proximal end, sized and configured to provide a yaw swivel freedom of about ±2.09 rad.;
d) a pair of coaxial horizontal bores defined within the apically flaring open portion;
e) a shelf portion extending proximally from the shelf, defining an aperture therein, the aperture being coaxial with the aperture defined by the partially circular extension, wherein the aperture defined in the shelf portion extending proximally from the shelf, and the aperture defined in the aperture defined by the partially circular extension of the second jaw are configured to receive a third hinging element; and
f) the third hinging element, pivotally coupling the dorsal hinge plateau, the shelf of the second jaw, the coaxial aperture extending through the basal surface of the partially circular extension of the second jaw, and the ventral hinge plateau, and slidably coupling a portion of a camming member.

According to some embodiments, the first jaw comprises:
a) a head portion having a distal end;
b) a rear section extending proximally from the head portion, the rear section sized and configured to be partially accommodated within the opening defined by the apically flaring open portion, wherein the rear section is comprised of two lobes, each lobe defining a through hole, sized and configured to pivotally couple to the apically flaring open portion of the second jaw and a circular depression.

According to some embodiments, the end effector actuating assembly comprises:
a. a fulcrum member coupled to the ventral opening in the sleeve;
b. the camming member having a proximal end and a frusto-spheroid distal end coupled to the circular depression defined in each of the lobes, pivotally coupled to the fulcrum member; and
c. a shuttle assembly, having a proximal end coupled to the distal end of the rod assembly, the shuttle assembly slidably coupled to the camming member, wherein translation of the shuttle assembly distally within the elongated interrupted cannula is configured to cause a frusto-spheroid distal end of the camming member to close the first jaw, and wherein translation of the shuttle assembly proximally within the elongated interrupted cannula is configured to cause a distal end of the camming member to open the first jaw

According to some embodiments, wherein the fulcrum member comprises:
a. a base plate having a flat apical surface and an arcuate basal surface, the arcuate basal surface having a curvature configured to complement the curvature of the sleeve adjacent to the ventral opening, defined toward the distal end of the sleeve, the base plate further defining a pair of slits, each slit configured to receive a portion of a fulcrum bracket; and
b. a pair of the fulcrum brackets separated by a predetermined gap, each fulcrum bracket having a proximal end and a distal end, a basal extension, and a partially circular portion extending dorsally from a proximal shelf and a distal shelf, defining an aperture, configured to receive a fulcrum hinge, wherein each of the basal extensions is configured to be accommodated and engaged within the corresponding slit defined in the base plate.

According to some embodiments, the camming member having a proximal end, a proximal portion having an upper surface, a mid-section, a distal portion having an upper surface, and the frusto-spheroid distal end, wherein:
a. the frusto-spheroid distal end further defines a bore having a rounded rectangle cross section, configured to accommodate the third hinging element;
b. the proximal portion further defines an arcuate basal surface;
c. the distal portion further defines an arcuate basal surface symmetric to the arcuate basal surface defined in the proximal portion of the camming member; and
d. the mid-section defining:
   i. a partially circular extension defining a co-axial bore;
   ii. an arcuate rim terminating in a proximal edge and a distal edge formed on both sides of the circular extension, the arcuate rim having a curvature that is complimentary to the curvature defined by the partially circular portion of each fulcrum member extending dorsally, wherein the partially circular extension having a thickness sized to be accommodated in the gap predefined between the pair of fulcrum members, and wherein the co-axial bore is configured to accommodate the fulcrum hinge.

According to some embodiments, the shuttle assembly comprises:
a. a proximal coupler, extending proximally from the proximal end of the shuttle
b. an elongated cylindrical body having a distal end defining a longitudinal axis extending distally from the proximal end of the shuttle;
c. a pair of partially cylindrical tines extending distally from the distal end of the elongated cylindrical body and terminating distally with a common distal end;
d. a proximal beveled rib coupling the pair of partially cylindrical tines, the proximal beveled rib disposed between a pair of co-axial proximal apertures defined in each tine and is sized and configured to partially accommodate a proximal bearing roller, spanning the gap between the pair of partially cylindrical tines; and
e. a distal beveled rib coupling the pair of partially cylindrical tines, the distal beveled rib disposed between a pair of co-axial distal apertures defined in each tine and is sized and configured to partially accommodate a distal bearing roller, spanning the gap between the pair of partially cylindrical tines.

According to some embodiments, the proximal bearing roller is configured to abut the arcuate basal surface of the proximal portion of the camming member, and wherein the distal bearing roller is configured to abut the arcuate basal surface of the distal portion of the camming member.

According to some embodiments, each of the arcuate basal surface of the proximal portion, and the arcuate basal surface of the proximal portion define an apogee configured such that:
a. when the distal bearing roller is beneath the apogee of the arcuate basal surface of the distal portion, the proximal bearing roller abuts the arcuate basal surface of the proximal portion, causing the frusto-spheroid distal end of the camming member to extend basally, and consequently the first jaw to open independently of a yaw swivel angle; and
b. when the proximal bearing roller is beneath the apogee of the arcuate basal surface of the proximal portion, the distal bearing roller abuts the arcuate basal surface of the distal portion, causing the frusto-spheroid distal end of the camming member to extend apically, and consequently the first jaw to close independently of a yaw swivel angle.

According to an aspect of some embodiments, there is provided a surgical instrument comprising a swiveling end effector having a first jaw and a second jaw, the first jaw movable between an open position and a closed position partially abutting the second jaw, the surgical instrument operable to deliver a predetermined torque for closing the first jaw that is independent of a yaw swivel angle.

According to some embodiments, the swiveling end effector is a punch, a grasper, a suture passer, bypass scissors, anvil scissors, or an end effector having the first jaw and the second jaw. According to some embodiments, the surgical instrument further comprises:
a. an elongated cylindrical shaft defining a longitudinal axis, having a distal end operably coupled to the swiveling end effector, and a proximal end operably coupled to a stationary handle;
b. the stationary handle;
c. an articulating handle, hingedly coupled to the stationary handle and operably coupled to a first jaw in the swiveling end effector, configured to operate the swiveling end effector; and
d. a swiveling member, operably coupled to a second jaw in the swiveling end effector, operable to swivel the swiveling end effector to a yaw swivel angle of about ±2.09 radians (rad.) from the longitudinal axis, wherein the second jaw partially abuts the first jaw.

According to some embodiments, the shaft comprises:
a. a sleeve, having a proximal end coupled to the stationary handle, and a distal end coupled to a ventral coupling member and a dorsal coupling member;
b. an elongated cannula nested within- and coaxial with the sleeve, having a proximal end operably coupled to the swiveling member, and a distal end having a partially cylindrical extension defining a vertical partial bevel gear at its distal end; and
c. a rod assembly, nested within the elongated cannula, the rod assembly having a proximal end operably coupled to the articulating handle, and a distal end coupled to an end effector actuating assembly.

According to some embodiments, the second jaw having a distal end and a proximal end, further comprises:
a. a partially circular extension extending proximally from the proximal end of the second jaw, the partially circular extension having a basal surface and an apical surface; and
b. a horizontal partial beveled gear, or face gear, extending apically from the apical surface of the partially circular extension, the horizontal partial beveled gear defining a coaxial aperture extending through the basal surface of the partially circular extension, wherein the horizontal partial beveled gear is configured to engage the vertical partial bevel gear at the distal end of the elongated cannula;
c. an apically flaring open portion, forming a spilt shelf defining a groove sized and configured to accommodate a portion of the first jaw, the open portion defining a proximal end, sized and configured to provide a yaw swivel freedom of about ±2.09 rad.;
d. a pair of coaxial horizontal bores defined within the apically flaring open portion transverse to the groove defined by the split shelf;
e. a dorsal hinge member, operably coupled apically to the split shelf, having a portion extending proximally from the shelf, defining an aperture therein, the aperture being coaxial with the aperture defined by the horizontal partial beveled gear coupled to apical surface of the partially circular extension.

According to some embodiments, the first jaw comprises:
a. a head portion having a distal end;
b. a lever having a front section, a mid-section, and a rear section extending proximally from the head portion, the front section and mid-section adapted sized and configured to be partially accommodated within the groove defined by the apically flaring open portion,
wherein:
   i. the mid-section further defines a through hole, sized and configured to pivotally couple to the apically flaring open portion of the second jaw; and
   ii. the rear section defining a circular depression.

According to some embodiments, the end effector actuating assembly further comprises:
a. a camming element, having an L-shape with a first leg having a ball joint disposed at the distal end of the first leg, while the proximal end of the first leg is coupled to a second leg having a distal end defining a through hole, transverse to the second leg, and a proximal end, terminating in a pair of lateral extensions forming a gap there between, each lateral extension defining a pair of coaxial apertures disposed toward the proximal end of each of the pair of lateral extensions, wherein contour of the circular depression defined in the rear section of the lever extending proximally from the head portion of the second jaw is complimentary to the ball joint, and is configured to abut a portion of the ball joint;
b. a pair of slider slabs, each having a narrow distal facet that is larger than an opposing narrow proximal facet, with a slider slab aperture defined through the slab in proximity to the basal portion of the slider slab towards the distal facet, and a tab extending from the upper narrow facet of the slab, the tab having a distal wall and a proximal wall;
c. a first hinging element, pivotally coupling the pair of slider slabs to the camming element via the through hole defined in the distal end of the second leg of the L-shape camming element; and
d. a second hinging element, pivotally coupling the second jaw and the first jaw by extending through the through hole defined in the mid-section of the lever extending proximally from the head portion of the first jaw, disposed within the groove defined by the split shelf and being coaxial with the pair of coaxial horizontal bores defined within the apically flaring open portion transverse to the groove.

According to some embodiments, the end effector actuating assembly further comprises:
a. an elongated slider having a proximal end coupled to the distal end of the rod assembly, and a distal end, the slider further having an upper surface defining a channel having a sloped portion and a horizontal portion, the upper surface configured to partially accommodate a complementary basal surface of a camming arm, wherein the distal end further includes a pair of extensions extending distally, defining an axial groove adapted sized and configured to partially accommodate a coupling slab, each of the pair of extensions further defines a coaxial horizontal bores transverse to the axial groove;
b. the coupling slab having an arcuate distal end and an arcuate proximal end, the diameter defined by the arcuate proximal end being larger than the diameter defined by the arcuate proximal end, the coupling slab defining a distal through hole and a proximal through hole;
c. the camming arm having a proximal end and a frusto-spheroid distal end, a distal portion and a proximal portion, wherein the distal portion further defines a cavity configured to accommodate the arcuate distal end of the coupling slab, the distal portion further defines a pair of apertures sized, adapted and configured coaxially with the distal through hole defined in the arcuate distal end of the coupling slab, and wherein the proximal portion further comprise a drill hole defined towards the proximal end;
d. a pair of bracket slabs, each having a narrow distal facet and an opposing narrow proximal facet, with a bracket slab aperture defined through the slab in proximity to a proximal end of the slider bracket, and a tab extending apically from the upper narrow facet of the bracket slab, the tab having a distal wall and a proximal wall;
e. a seventh hinging element, pivotally coupling the pair of slider brackets to the drill hole defined towards the proximal end of the camming arm by extending through the bracket slab aperture defined through each bracket slab in proximity to a proximal end of the bracket slab;
f. an eighth hinging element, pivotally coupling the second jaw and the first jaw by extending through the through hole defined in the mid-section of the lever extending proximally from the head portion of the first jaw, disposed within the groove defined by the split shelf and being coaxial with the pair of coaxial horizontal bores defined within the apically flaring open portion transverse to the groove;
g. a ninth hinging element, pivotally coupling the coupling slab and the camming arm, by extending through the pair of apertures defined in the distal portion of the camming arm and the coaxial distal through hole defined in the arcuate distal end of the coupling slab accommodated in the cavity defined in the distal portion of the camming element; and
h. a tenth hinging element, pivotally coupling the elongated slider and the coupling slab, by extending through the coaxial horizontal bores transverse to the axial groove defined by the pair of extensions extending distally from the elongated slider, defining the axial groove adapted sized and configured to partially accommodate the arcuate proximal end of the coupling slab, and the coaxially disposed proximal through hole.

According to some embodiments, the swiveling end effector further comprises:
a. a dorsal coupling member, coupled to- and extending distally from the distal end of the shaft, the dorsal coupling member comprising:
   i. a distal portion, defining a dorsal aperture, the dorsal aperture being coaxial with the aperture defined in the dorsal hinge member portion extending proximally from the split shelf of the second jaw; and
ii. a proximal portion, defining a rectangular window, the rectangular window's narrow aspect sized and configured to accommodate the pair of lateral extensions of the second leg of the camming element, sandwiched between the pair of tabs flaring apically from each slider slabs;
b. a ventral coupling member, coupled to- and having a distal portion extending distally from the distal end of the shaft, the ventral coupling member comprising a distal portion defining a ventral aperture being coaxial with the coaxial aperture of the horizontal partial beveled gear, extending through the basal surface of the partially circular extension of the second jaw; and
c. a third hinging element, pivotally coupling the dorsal hinge member coupled to the split shelf of the second jaw extending proximally, and the distal portion of the dorsal coupling member via extending through the aperture defined in the distal portion of the dorsal coupling member, and the aperture defined in the dorsal hinge member: and
d. a fourth hinging element, pivotally coupling the ventral coupling member and the basal surface of the partially circular extension of the second jaw, by extending through the ventral aperture of the ventral coupling member, and the coaxial aperture of the horizontal partial beveled gear, extending through the basal surface of the partially circular extension.

According to some embodiments, the swiveling end effector further comprises:
a. a dorsal coupling member, coupled to- and extending distally from the distal end of the shaft, the dorsal coupling member comprising:
   i. a distal portion, defining a dorsal aperture, the dorsal aperture being coaxial with the aperture defined in the dorsal hinge member portion extending proximally from the split shelf of the second jaw; and
   ii. a proximal portion, defining a rectangular window, the rectangular window's narrow aspect sized and configured to accommodate the pair tabs extending apically from the upper narrow facet of each of the bracket slab, and the proximal portion of the camming arm sandwiched between the pair of tabs;
b. a ventral coupling member, coupled to- and having a distal portion extending distally from the distal end of the shaft, the ventral coupling member comprising a distal portion defining a ventral aperture being coaxial with the coaxial aperture of the horizontal partial beveled gear, extending through the basal surface of the partially circular extension of the second jaw; and
c. a third hinging element, pivotally coupling the dorsal hinge member coupled to the split shelf of the second jaw extending proximally, and the distal portion of the dorsal coupling member via extending through the aperture defined in the distal portion of the dorsal coupling member, and the aperture defined in the dorsal hinge member: and
d. a fourth hinging element, pivotally coupling the ventral coupling member and the basal surface of the partially circular extension of the second jaw, by extending through the ventral aperture of the ventral coupling member, and the coaxial aperture of the horizontal partial beveled gear, extending through the basal surface of the partially circular extension.

According to some embodiments, the stationary handle further comprises a head portion having a distal end and a proximal end, and a body portion, wherein the head portion defines:
a. a sleeve bore coaxial with the longitudinal axis of the shaft, the sleeve bore having a distal end and a proximal end;
b. a first cavity expanding from the proximal end of the sleeve bore, the first cavity adapted, sized and configured to accommodate a portion of the swiveling ratchet; and
c. a second cavity, adapted sized and configured to accommodate a portion of the articulating handle and a portion of the rod assembly.

According to some embodiments, the head portion further defines:
a. a hinge aperture configured to receive a sixth hinging element;
b. a proximal slot extending through the proximal end of the head portion, through the hinge aperture, to the second cavity; and
c. a partially circular ventral slot, flaring apically to the first cavity.

According to some embodiments, the articulating handle comprises a linking member, flaring apically from the distal end of the articulating handle, the linking member sized and configured to be accommodated in the proximal slot of the head portion of the stationary handle, further defining an apical protrusion, operable to pivotally couple to a proximal member, and wherein the linking member is hingedly coupled to the stationary handle via a sixth hinging element.

According to some embodiments, the rod assembly comprises:
a. a push-rod having a proximal end and a distal end
b. the proximal member extending proximally from the proximal end of the push rod; and
c. a camming linker, flaring apically and distally at a predetermined angle from the distal end of the push rod, the camming linker is sized to be accommodated within the gap formed by pair of lateral extensions of the camming element, and is operable to hingedly couple to the camming element via a fifth hinging element extending through the pair of coaxial apertures disposed toward the proximal end of each of the pair of lateral extensions and the camming linker sandwiched there between.

According to some embodiments, the swiveling member comprises:
a. a basal body portion defining a right depression and a left depression;
b. a flat neck portion, sized and configured to be accommodated within the partially circular ventral slot of the head portion of the stationary handle;
c. a hollow cylinder extending transverse to the neck portion, wherein the hollow cylinder is configured to couple to the proximal end of the elongated cannula; and
d. an arcuate indexing teeth portion disposed in parallel with the flat neck portion and transverse to the longitudinal axis defined by the hollow cylinder.

According to some embodiments, the first cavity further defines a radial channel configured to accommodate the arcuate indexing teeth portion, the first cavity further comprising a resilient tab, disposed transverse to-, and within, the radial channel, the resilient tab sized adapted and configured to releasably engage a tooth in the arcuate indexing teeth portion.

According to some embodiments, the hollow cylinder of the swiveling member has the same outer diameter as the shaft sleeve.

According to some embodiments, the elongated cannula further comprises a ventral opening disposed toward the proximal end of the elongated cannula.

According to some embodiments, the stationary handle further comprises a head portion having a distal end and a proximal end, and a body portion, wherein the head portion defines:
a. a sleeve bore coaxial with the longitudinal axis of the shaft, the sleeve bore having a distal end and a proximal end, the sleeve bore being adapted, sized and configured to accommodate a portion of the swiveling member; and
b. a cavity, adapted sized and configured to accommodate a biasing element and an indexing bearing.

According to some embodiments, the head portion further defines:
a. a hinge aperture configured to receive a sixth hinging element;
b. a slot extending through a basal surface of the head portion, and through the hinge aperture, to the sleeve bore; and
c. a partially circular dorsal slot, extending to the first cavity.

According to some embodiments, the rod assembly comprises:
a. a push-rod having a proximal end and a distal end; and
b. a camming linker, flaring apically and distally at a predetermined angle from the distal end of the push rod, the camming linker is sized to be accommodated within the gap formed by pair of lateral extensions of the camming element, and is operable to hingedly couple to the camming element via a fifth hinging element extending through the pair of coaxial apertures disposed toward the proximal end of each of the pair of lateral extensions and the camming linker sandwiched there between.

According to some embodiments, the articulating handle configured to be accommodated in the slot extending through a basal surface of the head portion of the stationary handle, further defining an apical protrusion, operable to pivotally couple to the proximal end of the push-rod, and wherein the apical protrusion is hingedly coupled to the stationary handle via a sixth hinging element.

According to some embodiments, the swiveling member comprises:
a. a body portion defining a right depression and a left depression;
b. a flat neck portion coupled to the body portion, defining a partially circular surface, coupled to the proximal end of the elongated cannula, the flat neck portion sized and configured to be accommodated within the partially circular dorsal slot of the head portion of the stationary handle, wherein the partially circular surface further defines an arcuate array of indexing holes or indexing depressions.

According to some embodiments, the first cavity further comprises the biasing element, operable to bias the bearing to engage one of the indexing holes or indexing depressions defined in the partially circular surface of the flat neck portion of the swiveling member.

According to some embodiments, the swiveling end effector is configured to provide a compression load of between about 29.42 newton and about 98.00 newton at any yaw swivel angle between the second jaw and the first jaw.

According to some embodiments, the shaft has an outer diameter that is no more than 4.5 mm.

According to some embodiments, the punch is an upbiter curved at an angle of between about 1° and about 15° upward at the tip.

According to some embodiments, the shaft is further swept up at a predetermined curvature.

According to some embodiments, the predetermined curvature is adapted and sized to follow a contour of the condyle, configured to maintain a lower surface of the first jaw in parallel with a tibial plateau.

According to some embodiments, the shaft comprises:
a. a sleeve, having a proximal end coupled to the stationary handle, and a flaring distal end hingedly coupled to the end effector, wherein the sleeve further defining:
   i. a ventral opening, defined toward the distal end of the sleeve, configured to accommodate a portion of an end effector actuating assembly;
   ii. a dorsal hinging plateau defined in the distal end of the sleeve; and
   iii. a ventral hinging plateau defined in the distal end of the sleeve;
b. an elongated interrupted cannula nested within- and coaxial with the sleeve, having a proximal end operably coupled to the swiveling member, and a distal end defining a horizontal spur gear at its distal end; and
c. a rod assembly, nested within the elongated cannula, the rod assembly having a proximal end operably coupled to the articulating handle, and a distal end coupled to the end effector actuating assembly.

According to some embodiments, the second jaw having a distal end and a proximal end, further comprising:
a. a partially circular extension extending proximally from the proximal end of the second jaw, the partially circular extension having a basal surface and an apical surface; and
b. a horizontal partial spur gear, extending from the partially circular extension, the horizontal partial spur gear defining a coaxial aperture extending through the basal surface of the partially circular extension, wherein the horizontal partial spur gear is configured to engage the partial spur gear at the distal end of the elongated cannula;
c. an apically flaring open portion, forming a shelf defining an opening sized and configured to accommodate a portion of the first jaw, the open portion defining a proximal end, sized and configured to provide a yaw swivel freedom of about ±2.09 rad.;
d. a pair of coaxial horizontal bores defined within the apically flaring open portion;
e. a shelf portion extending proximally from the shelf, defining an aperture therein, the aperture being coaxial with the aperture defined by the partially circular extension, wherein the aperture defined in the shelf portion extending proximally from the shelf, and the aperture defined in the aperture defined by the partially circular extension of the second jaw are configured to receive a third hinging element; and
f. the third hinging element, pivotally coupling the dorsal hinge plateau, the shelf of the second jaw, the coaxial aperture extending through the basal surface of the partially circular extension of the second jaw, and the ventral hinge plateau, and slidably coupling a portion of a camming member.

According to some embodiments, the first jaw comprises:
a. a head portion having a distal end;
b. a rear section extending proximally from the head portion, the rear section sized and configured to be partially accommodated within the opening defined by the apically flaring open portion, wherein the rear section is comprised of two lobes, each lobe defining a through hole, sized and configured to pivotally couple to the apically flaring open portion of the second jaw and a circular depression.

According to some embodiments, the end effector actuating assembly comprises:
a. a fulcrum member coupled to the ventral opening in the sleeve;
b. the camming member having a proximal end and a frusto-spheroid distal end coupled to the circular depression defined in each of the lobes, pivotally coupled to the fulcrum member; and
c. a shuttle assembly, having a proximal end coupled to the distal end of the rod assembly, the shuttle assembly slidably coupled to the camming member, wherein translation of the shuttle assembly distally within the elongated interrupted cannula is configured to cause a frusto-spheroid distal end of the camming member to close the first jaw, and wherein translation of the shuttle assembly proximally within the elongated interrupted cannula is configured to cause a distal end of the camming member to open the first jaw

According to some embodiments, the fulcrum member comprises:
a. a base plate having a flat apical surface and an arcuate basal surface, the arcuate basal surface having a curvature configured to complement the curvature of the sleeve adjacent to the ventral opening, defined toward the distal end of the sleeve, the base plate further defining a pair of slits, each slit configured to receive a portion of a fulcrum bracket; and
b. a pair of the fulcrum brackets separated by a predetermined gap, each fulcrum bracket having a proximal end and a distal end, a basal extension, and a partially circular portion extending dorsally from a proximal shelf and a distal shelf, defining an aperture, configured to receive a fulcrum hinge, wherein each of the basal extensions is configured to be accommodated and engaged within the corresponding slit defined in the base plate.

According to some embodiments, the camming member has a proximal end, a proximal portion having an upper surface, a mid-section, a distal portion having an upper surface, and the frusto-spheroid distal end, wherein:
a. the frusto-spheroid distal end further defines a bore having a rounded rectangle cross section, configured to accommodate the third hinging element;
b. the proximal portion further defines an arcuate basal surface;
c. the distal portion further defines an arcuate basal surface symmetric to the arcuate basal surface defined in the proximal portion of the camming member; and
d. the mid-section defining:
   i. a partially circular extension defining a co-axial bore;
   ii. an arcuate rim terminating in a proximal edge and a distal edge formed on both sides of the circular extension, the arcuate rim having a curvature that is complimentary to the curvature defined by the partially circular portion of each fulcrum member extending dorsally, wherein the partially circular extension having a thickness sized to be accommodated in the gap predefined between the pair of fulcrum members, and wherein the co-axial bore is configured to accommodate the fulcrum hinge.

According to some embodiments, the shuttle assembly comprises:
a, a proximal coupler, extending proximally from the proximal end of the shuttle
b. an elongated cylindrical body having a distal end defining a longitudinal axis extending distally from the proximal end of the shuttle;
c. a pair of partially cylindrical tines extending distally from the distal end of the elongated cylindrical body and terminating distally with a common distal end;
d. a proximal beveled rib coupling the pair of partially cylindrical tines, the proximal beveled rib disposed between a pair of co-axial proximal apertures defined in each tine and is sized and configured to partially accommodate a proximal bearing roller, spanning the gap between the pair of partially cylindrical tines; and
e. a distal beveled rib coupling the pair of partially cylindrical tines, the distal beveled rib disposed between a pair of co-axial distal apertures defined in each tine and is sized and configured to partially accommodate a distal bearing roller, spanning the gap between the pair of partially cylindrical tines.

According to some embodiments, the proximal bearing roller is configured to abut the arcuate basal surface of the proximal portion of the camming member, and wherein the distal bearing roller is configured to abut the arcuate basal surface of the distal portion of the camming member.

According to some embodiments, each of the arcuate basal surface of the proximal portion, and the arcuate basal surface of the proximal portion define an apogee configured such that:
a. when the distal bearing roller is beneath the apogee of the arcuate basal surface of the distal portion, the proximal bearing roller abuts the arcuate basal surface of the proximal portion, causing the frusto-spheroid distal end of the camming member to extend basally, and consequently the first jaw to open independently of a yaw swivel angle; and
   b. when the proximal bearing roller is beneath the apogee of the arcuate basal surface of the proximal portion, the distal bearing roller abuts the arcuate basal surface of the distal portion, causing the frusto-spheroid distal end of the camming member to extend apically, and consequently the first jaw to close independently of a yaw swivel angle. Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE SEVERAL VIEWS OF THE DRAWINGS

Surgical instruments each having a swiveling end effector configured to deliver the same load at any yaw angle relative to a longitudinal axis of the surgical instrument are disclosed herein, and will become apparent from the following detailed description when read in conjunction with the figures, which are exemplary, and are not intended to be limiting.

Some embodiments of the invention are herein described, by way of example only, with reference to the accompanying drawings and images. With specific reference now to the drawings and images in detail, it is stressed that the particulars shown are by way of example and for purposes of illustrative discussion of embodiments of the invention. In this regard, the description taken with the drawings and images makes apparent to those skilled in the art how embodiments of the invention may be practiced.

In the Drawings and Images:

FIGS. 1-2 are respective front left and front right perspective views of an exemplary implementation of the surgical instrument with a punch end-effector, according to some embodiments;

FIG. 3 is a cut-away schematic enlargement of inset D illustrated in FIG. 1, showing a portion of the surgical instrument, according to some embodiments;

FIG. 4A is an enlargement of inset D illustrated in FIG. 1 with

FIG. 4B illustrating an exemplary implementation of a camming element of the surgical instrument, according to some embodiments;

FIG. 5A is a schematic illustrating the distal portion of the actuation mechanism of the first jaw with

FIG. 5B illustrating an exemplary implementation of a slider slab element, according to some embodiments.

FIG. 6A is a schematic illustration of the distal swiveling mechanism of the second jaw with

FIG. 6B, illustrating a top rear right perspective of the second jaw and

FIG. 6C, illustrating a portion of a second jaw actuation mechanism, including a vertical partial beveled gear, according to some embodiments;

FIG. 7A is a partial cross sectional view taken along lines A-A in FIG. 1, according to some embodiments with

FIG. 7B illustrating the proximal configuration of the articulating handle and the swiveling member in reference to the shaft, according to some embodiments, and

FIG. 7C is a cross sectional view of the surgical instrument taken along lines B-B in FIG. 1, according to some embodiments.

FIG. 8 is a cross sectional view of the surgical instrument's shaft taken along lines C-C in FIG. 1, according to some embodiments;

FIG. 9A is a rear perspective view of the articulating handle in relation to the stationary handle, according to some embodiments, with

FIG. 9B being a perspective view of the swiveling member in relation to the stationary handle, according to some embodiments.

FIG. 10 is a front left perspective view of an exemplary implementation of the surgical instrument with a grasper end-effector, according to some embodiments;

FIG. 11 is an enlargement of inset E of the surgical instrument illustrated in FIG. 10, according to some embodiments;

FIGS. 12A-B are exploded views of selected components in the enlarged portion illustrated in FIG. 11, according to some embodiments, with

FIG. 12C illustrating a portion of a surgical instrument having an alternative swiveling assembly including a camming arm, according to some embodiments;

FIG. 13A is a rear left perspective view of an articulating handle in an exemplary implementation in relation to the stationary handle, according to some embodiments, with

FIG. 13B, being a front perspective view of the swiveling member in relation to the stationary handle, according to some embodiments;

FIG. 14A is a partial cross sectional view of a portion of the surgical instrument shown in FIG. 10, taken along lines F-F in FIG. 10, according to some embodiments, with

FIG. 14B being is a partial cross sectional view of the portion of the surgical instrument shown in FIG. 14A, where the articulating handle has been removed for clarity, according to some embodiments;

FIGS. 15A, 15B, 15C, and 15D illustrate alternative end effectors, according to some embodiments;

FIG. 16A illustrates an exemplary surgical instrument having an end effector assembly, according to some embodiments, with FIG. 16B illustrating the foregoing at a different perspective, while FIG. 16C illustrates another exemplary implementation of a second jaw, according to some embodiments, and FIG. 16D illustrates a cut-out side view of a portion of the surgical instrument of FIG. 16B, according to some embodiments;

FIG. 17A illustrates a portion of a surgical instrument having an end effector actuating assembly, according to some embodiments, with FIG. 17B being a Y-Z cross sectional view of the surgical instrument, taken along line 17B-17B in FIG. 16D, according to some embodiments;

FIG. 18A illustrates the distal end of an elongated cannula of the surgical instrument of FIG. 16A, according to some embodiments, with

FIG. 18B, illustrating a portion of the end effector actuating assembly, according to some embodiments;

FIG. 19 illustrates an exploded view of an exemplary implementation of components of a surgical instrument having an end effector actuating assembly, according to some embodiments;

FIG. 20 illustrates an exemplary implementation of the shuttle assembly, which may be included in a surgical instrument having a swiveling end effector, according to some embodiments;

FIG. 21, illustrates an exemplary implementation of a camming member, which may be included in a surgical instrument having a swiveling end effector, according to some embodiments;

FIG. 22A illustrates a more detailed view of an end effector actuating assembly depicting closed jaws, according to some embodiments, with FIG. 22B illustrating a more detailed view of the end effector actuating assembly with the jaws open, according to some embodiments;

FIG. 23A is a sectional view of a portion of a surgical instrument, showing the distal portion of a sleeve, illustrating an exemplary implementation of the fulcrum member, according to some embodiments, with FIG. 23B illustrating an exemplary implementation of the fulcrum member, according to some embodiments;

FIG. 24 is a schematic illustration of a kit of surgical instruments that may be used in a single operation, according to some embodiments; and

FIG. 25 illustrates an example where bite force was measured at different yaw angles, according to some embodiments.

While the disclosure of the surgical instrument provided herein is amenable to various modifications and alternative forms, specifics thereof have been shown by way of example only in the drawings and will be further described in detail hereinbelow. It should be understood, however, that the intention is not to limit the disclosure to the particular exemplary implementations described. On the contrary, the intention is to cover all modifications, equivalents, and alternatives.

### DETAILED DESCRIPTION

Provided herein are exemplary implementations of a surgical instrument comprising a swiveling end effector operable to deliver a predetermined torque for closing a jaw that is independent of a yaw swivel angle. The instrument can be used for example in the repair of torn menisci, or in any other surgical procedure, as discussed herein.

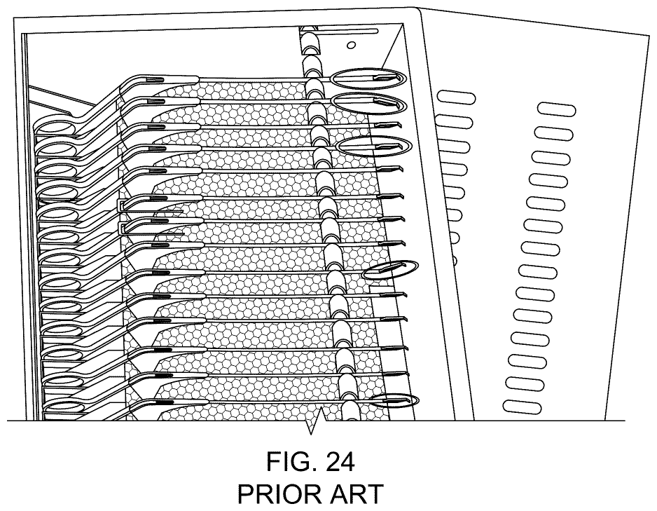

Meniscal tears are the most frequently encountered and treated injuries in the knee joint, in both young, active sports people and in elderly people, and with a relatively high annual cost. Similarly, meniscal tear surgery is among the most commonly performed type of procedure in orthopaedic surgery. Three main methods for the surgical management of meniscus tears have been used and include: meniscectomy, meniscal repair, and meniscal reconstruction. The disclosed surgical instrument can be used in any arthroscopic or open surgery in the management of this pathology. Some of the typical instruments previously in use for such arthroscopic or open surgery are depicted in FIG. 24.

In addition, and as needed, the surgical instrument disclosed can be used in other arthroscopic surgeries as well.

An aspect of some embodiments relates to a surgical instrument having a swiveling end effector. In some embodiments of the invention, the surgical instrument comprises a swiveling end effector having a first jaw and a second jaw, the first jaw movable between an open position and a closed position at least partially abutting the second jaw, by application of force to at least one handle located at a proximal portion of the surgical instrument. The swiveling end effector is also configured to swivel about a swivel axis perpendicular to a shaft of the surgical instrument. The surgical instrument is operable to deliver a predetermined torque for closing the first jaw that is affected less than 10% by a yaw swivel angle, as discussed further herein. In other words, when a certain amount of force is applied on the handle of the surgical instrument, an amount of torque is transmitted to the swiveling end effector, the amount of torque transmitted is not affected by the yaw swivel angle.

Movement of the end effector through multiple angles relative to the instrument shaft may be conventionally referred to as “articulation.” Articulation may be accomplished by a pivot (or articulation) joint being placed in the elongate shaft proximal to the end effector. This may allow the clinician to articulate the end effector remotely to either side for better and easier tissue manipulation and orientation. An articulating end effector may permit the clinician to more easily engage tissue in some instances, such as behind an organ. In addition, articulated positioning advantageously allows an endoscope to be positioned behind the end effector without being blocked by the elongate shaft.

An aspect of some embodiments relates to the provision of multiple swivel angles in a single end effector which may allow the same surgical instrument to be utilized at a number of angles in a single procedure, without the surgeon having to change instruments. This may simplify the procedure, in terms of time, ease of selecting an appropriate instrument, and trauma to the patient.

An aspect of some embodiments relates to a surgical instrument having an end effector at a distal end of the surgical instrument, the end effector configured to swivel about a swivel axis that is perpendicular to a longitudinal axis of the surgical instrument, the end effector configured to swivel in both directions, i.e., clockwise and counterclockwise about the swivel axis.

An aspect of some embodiments relates to articulation of an end effector of a surgical instrument, and pivoting or swiveling the end effector about a swivel axis which may be achieved by rotating the shaft to affect a change in the yaw angle. This may be advantageous in that the degree of freedom of the end effector in terms of the degree of yaw articulation may be increased, thereby increasing the flexibility of the device for the clinician. Further, such flexibility may be achieved since the load, or the force exerted by the jaws of the instrument, is not dependent on the swivel angle of the end effector. In other words, the force exerted by the jaws will not be changed with different selected swivel angles. It should be noted that the end effector is configured to pivot about a pivot axis which extends through the longitudinal axis of the shaft of the surgical instrument.

An aspect of some embodiments relates to a jaw design adapted for anatomy, for example the meniscus. In some embodiments, the lower jaw is shaped to facilitate interaction between the jaws of the end effector and tissue to which the end effector is applied. For example, an end effector having a narrower distal portion on the lower jaw may potentially allow the lower jaw to be more easily inserted beneath tissue such as, for example, a meniscus, thereby allowing better positioning of the jaws relative to the tissue.

An aspect of some embodiments relates to affecting a desired amount of end effector articulation and rotation by using only one hand. For example, many vascular operations require precise control of the end effector. In some embodiments there is provided a surgical instrument that employs a single control mechanism for selectively articulating and swiveling the end effector that can be easily actuated by using the same hand that is supporting the handle portion of the instrument. Further, the surgical instrument discussed herein has features which facilitate its use by a single hand of a right-handed or a left-handed clinician. For example, the thumb and middle finger of one hand may be used to actuate the closing of the jaws, while the index finger of the same hand may be used to swivel the swiveling end effector, as discussed herein.

An aspect of some embodiments relates to a swiveling end effector that may be removable from the surgical instrument and may be replaced by another end effector. For example, any of the end effectors shown and described herein may be replaced by another of the end effectors shown and described herein. Therefore, there may be provided a kit having a surgical instrument including a first end effector and at least one additional end effector. The end effectors may be any one of the end effectors described and shown herein, and additional end effectors having additional structures and functions, which may similarly replace an end effector in the surgical instrument, will be apparent to persons skilled in the art.

In an exemplary implementation, provided herein is a surgical instrument comprising a swiveling end effector having a first jaw and a second jaw, the first jaw movable between an open position and a closed position at least partially abutting the second jaw, the surgical instrument operable to deliver a predetermined torque for closing the first jaw against the second jaw that is independent of a yaw swivel angle.

In an exemplary implementation, provided herein is a surgical instrument comprising: a swiveling end effector operable to operate at a constant load at any yaw swivel angle; an elongated cylindrical shaft defining a longitudinal axis, having a distal end operably coupled to the swiveling end effector, and a proximal end operably coupled to a stationary handle; the stationary handle; an articulating handle, hingedly coupled to the stationary handle and operably coupled to a first jaw in the swiveling end effector, configured to operate the swiveling end effector; and a swiveling member (optionally including a ratchet mechanism), operably coupled to a second jaw in the swiveling end effector, operable to swivel the swiveling end effector to a yaw swivel angle of up to about 90 degrees relative to the longitudinal axis, according to some embodiments, wherein the swiveling end effector may be operable to transition between a closed position whereby the second jaw may be at least partially accommodated within and/or abuts the first jaw, and an open position whereby the distal end of the second jaw may be at a distance from the first jaw, according to some embodiments. According to some embodiments, the swiveling end effector is operable to swivel to a swivel angle of up to about 90 degrees, in either direction, relative to the longitudinal axis. According to some embodiments, the swiveling end effector is operable to swivel to a swivel angle of ±120 degrees.

An aspect of some embodiments relates to a surgical instrument having an end effector at a distal portion of the surgical instrument, and an actuator for transmitting force applied at a proximal portion of the surgical instrument to torque applied to the end effector, to close jaws of the end effector. The end effector is adapted to swivel about a swivel axis, and the amount of torque applied at the end effector is sufficient to close the jaws of the end effector.

An aspect of some embodiments relates to a surgical instrument having an end effector at a distal portion of the surgical instrument, and an actuator for transmitting force applied at a proximal portion of the surgical instrument to torque applied to the end effector, to close jaws of the end effector. The end effector is adapted to swivel about a swivel axis, and the amount of torque applied at the end effector when a force is applied at the surgical instrument proximal portion is substantially the same such as, for example, +30% or ±20% or ±10%, regardless of the swivel angle of the end effector.

An aspect of some embodiments relates to an arthroscopic surgical instrument having a longitudinal axis and an end effector including a biting tool configured to bite a meniscus in a knee, said biting tool having a first jaw and a second jaw, the first jaw manually actuatable to move between an open position and a closed position relative to the second jaw, the end effector configured swivel about a swivel axis which is perpendicular to a longitudinal axis of the arthroscopic surgical instrument.

Before explaining at least one embodiment of the invention in detail, it is to be understood that the invention is not necessarily limited in its application to the details of construction and the arrangement of the components and/or methods set forth in the following description and/or illustrated in the drawings and/or the Examples. The invention is capable of other embodiments or of being practiced or carried out in various ways.

### Definitions

The term “coupled,” including its various forms such as “operably coupling,” “coupling” or “couplable” refers to and may include any direct or indirect, structural coupling, connection or attachment, or adaptation or capability for such a direct or indirect structural or operational coupling, connection or attachment, including integrally formed components and components which are coupled via or through another component or by the forming process. Indirect coupling may involve coupling through an intermediary member or adhesive, or abutting and otherwise resting against, whether frictionally or by separate means without any physical connection.

In addition, for the purposes of the present disclosure, directional or positional terms such as “proximal,” “distal,” “top,” “bottom,” “upper,” “lower,” “side,” “front,” “frontal,” “forward,” “rear,” “rearward,” “back,” “trailing,” “above,” “below,” “left,” “right,” “radial ,” “vertical,” “upward,” “downward,” “outer,” “inner,” “exterior,” “interior,” “intermediate,” “apical,” “basal,” etc. are merely used for convenience in describing the various exemplary implementations of the present disclosure.

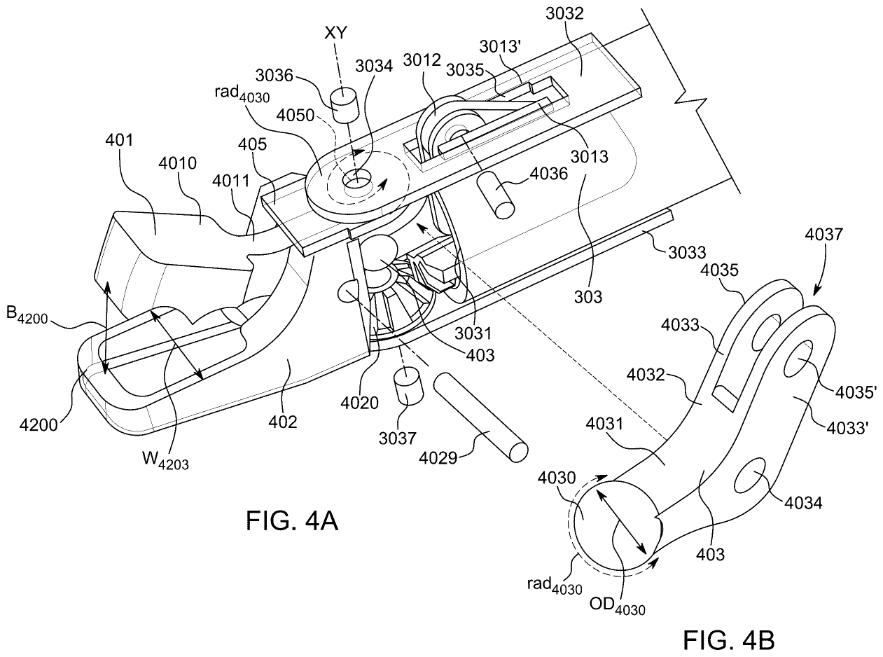

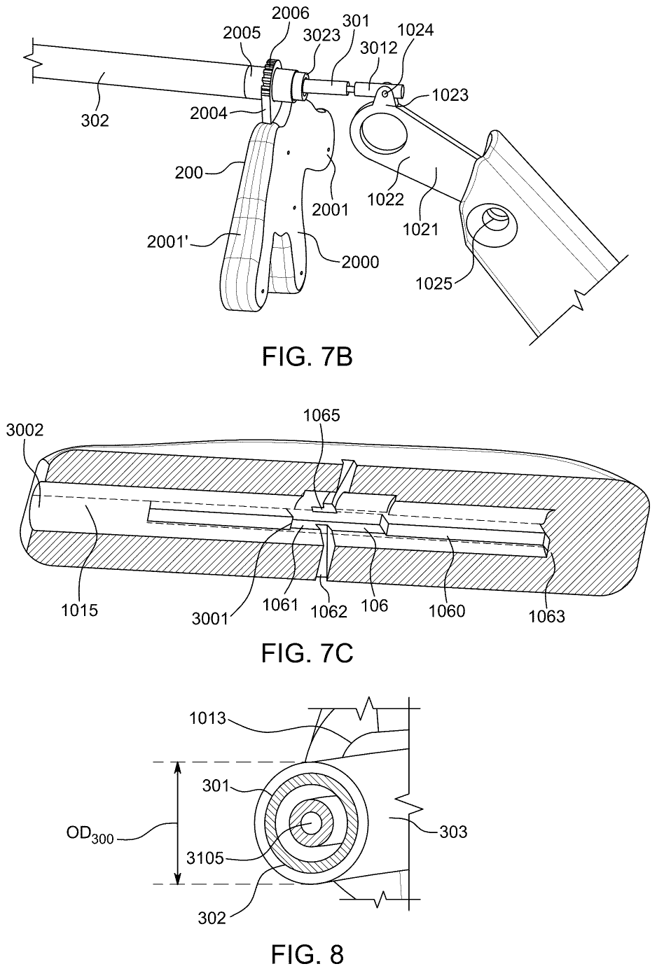

Likewise, the term “engage,” and various forms thereof, when used with reference to an engaging element, for example in the engagement of horizontal partial beveled gear 4021, and vertical partial beveled gear 3021 (see e.g., FIG. 3), refers in an exemplary implementation to the application of any forces that tend to hold horizontal partial beveled gear 4021 and vertical partial beveled gear 3021 together against inadvertent or undesired separating forces (e.g., such as may be introduced during swiveling of the end effector). It is to be understood, however, that engagement does not in all cases require an interlocking connection that is maintained against every conceivable type or magnitude of separating force. Further, the term “engaging element” refers in another exemplary implementation to one or a plurality of coupled components, at least one of which is configured for releasably engaging another element. Thus, this term encompasses both single part engaging elements and multi-part-assemblies, for example swiveling member 200 in cavity 106 as a whole (see e.g., FIGS. 7A, 7C).

The terms “first,” “second,” and the like, herein, do not denote any order, quantity, or importance, but rather are used to distinguish one element from another. The terms “a,” “an” and “the” herein do not denote a limitation of quantity, and are to be construed to cover both the singular and the plural, unless otherwise indicated herein or clearly contradicted by context. The suffix “(s)” as used herein is intended to include both the singular and the plural of the term that it modifies, thereby including one or more of that term such as, for example, lance-member(s) intended to include one or more lance member).

Reference throughout the specification to “one exemplary implementation,” “another exemplary implementation,” “an exemplary implementation,” and so forth means that a particular element (e.g., step, feature, structure, and/or characteristic) described in connection with the exemplary implementation is included in at least one exemplary implementation described herein, and may or may not be present in other exemplary implementations. In addition, it is to be understood that the described elements may be combined in any suitable manner in the various exemplary implementations.

In the context of the disclosure, the term “operable” means the system and/or the device, or a certain element or step is/are functional, sized, adapted and calibrated, comprises elements for, and meets applicable operability requirements to perform a recited function when activated, coupled, implemented, actuated, effected, or realized. In relation to systems, the term “operable” means the system is fully functional and calibrated, having the necessary elements, as well as the mechanisms for, and meets applicable operability requirements to perform a recited function when executed by a user.

The term “abut” refers in the context of the disclosure, to items that are in direct physical contact with each other, although the items may not be attached, secured, fused, glued, sewn, or welded together.

In the context of the disclosure, the term “accommodate” refers to the ability of an accommodating element (e.g., proximal slot 106 extending through the proximal end 1002 of head portion 1000, through hinge aperture 1025, to the second cavity 1060) to allow passage or retention of another element (e.g., linking member 1022 extending distally from distal end 1021 of articulating handle 102) at close tolerance, without substantial space for other elements or components.

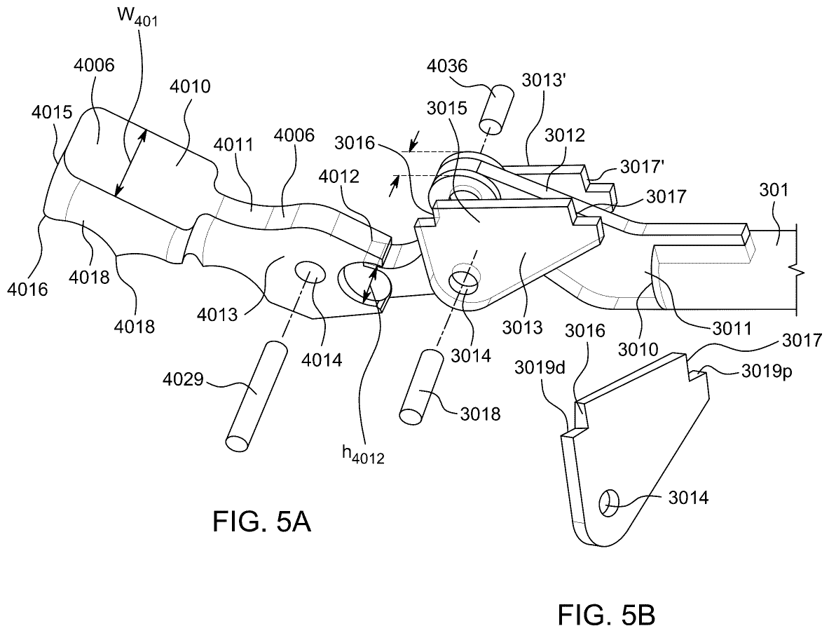

In the context of the disclosure, the term “L-shape” may be clarified by again referring to FIG. 4B. The camming element 403 contains leg 4031 that forms a distal edge to camming element 403. Leg 4031 may be oriented in a direction that may be at generally right angles to leg 4032

In the context of the disclosure, the term “C-shape” refers to any single structure that terminates in two prongs or legs the majority of which extend in a same general direction. Transition between such prongs or legs may be curved, or more of an acute right angle, such as shown.

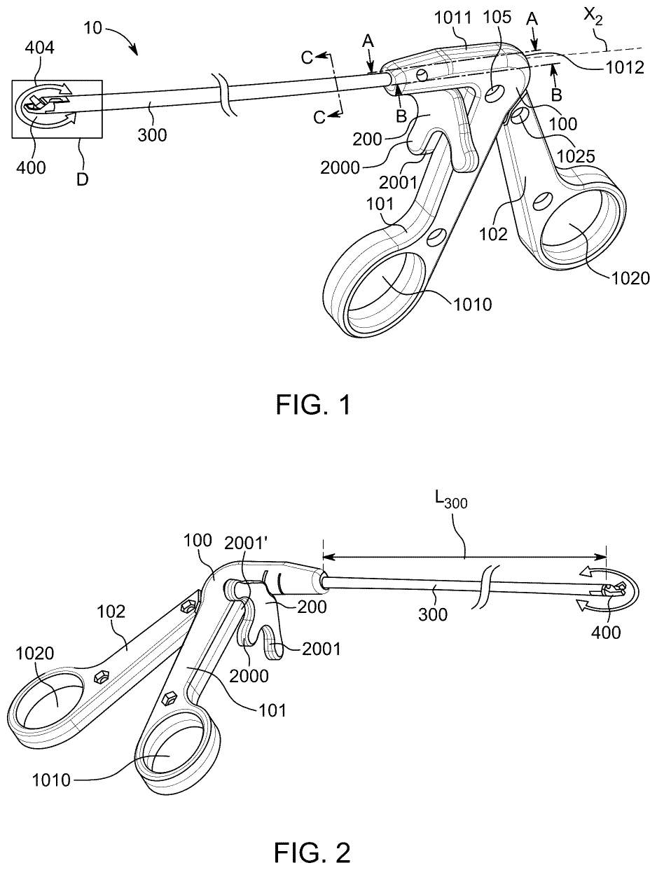

As used herein, the term “yaw” refers to an orientation of end effector 400 with respect to elongated cylindrical shaft 300 longitudinal axis XL (see e.g. FIG. 1). In the exemplary implementation, end effector 400 may be configured to selectively swivel with respect to elongated cylindrical shaft 300 about yaw axis (also referred to as “swivel axis”) Xy (see e.g., FIG. 4A). Furthermore, in the context of the disclosure, the term “swivel” also denotes, as is conventional, a rotational guidance about an axis (substantially without sliding, apart from functional clearance), for example the swivel linkage formed by the third hinging element 3036, the fourth hinging element 3037 and the center of the ball joint 4030, all of which are coaxial at any yaw swivel angle, imparting the consistent power to the end effector (see e.g., FIG. 4A).

The term “pivotally coupled” as used herein, means that two components are attached to each other, perhaps via one or more other components, such that one or both of the two components can rotate. Additionally, or alternatively, the term “pivotally coupled” refers to a situation where one element may be coupled to another element in a fixed spatial relation, but may be free to rotate with respect to the other element. In other words, no substantial lateral movements of the two elements take place, while relative rotation between the two elements may be possible. In yet other words, the term “pivotally coupled” refers to a situation where the rotation of the one element does not necessarily result in a rotation of the other element and vice versa. The one element may be supported with respect to or mounted to the other element in a way that permits rotation, such as via a pin, detent, hinge and the like.

Furthermore, in the context of the disclosure, the term “hinge” and its derivatives (e.g., “hinging element”) refer to one or more parts which allow the second component to pivot with respect to the first component. Accordingly, “hingedly coupled” indicates that the orientation of one component relative to the other can be varied. This may be because of a connecting region, which permits rotation, or because of a form of mechanical connection that permits relative movement, for example a pivot or a hinge pin. There may be an intermediate portion, which may be hinged at respective spaced locations to the first and second portions, allowing a greater degree of hinging in one or more directions.

In the context of the disclosure, “biasing element” refers to any element that provides a biasing force. Representative biasing elements include but are not limited to springs (e.g., elastomeric or metal springs, torsion springs, coil springs, leaf springs, tension springs, compression springs, extension springs, spiral springs, volute springs, flat springs, and the like), detents (e.g., spring-loaded detent balls, cones, wedges, cylinders, and the like), pneumatic devices, hydraulic devices, magnets, and the like, and combinations thereof. Likewise, “biasing member” as used herein refers to one or more members that apply an urging force between two elements.

In the context of the disclosure, the term “fulcrum” is meant to include but not limited to the center of balance of the camming member and/or the pivot point of the camming member and encompasses, for example, any pivotable connection or other support allowing the camming member to (partially) rotate.

A more complete understanding of the surgical instrument having a swiveling end effector configured to deliver the same load onto at least one jaw at any yaw angle relative to a longitudinal axis of the surgical instrument, can be obtained by reference to the accompanying drawings. These figures (also referred to herein as “FIG.”) are merely schematic representations based on convenience and the ease of demonstrating the present disclosure, and are, therefore, not intended to indicate relative size, scale and dimensions of the devices or components thereof, and/or to define or limit the scope of the exemplary implementations. Although specific terms are used in the following description for the sake of clarity, these terms are intended to refer only to the particular structure of the exemplary implementations selected for illustration in the drawings, and are not intended to define or limit the scope of the disclosure. In the drawings and the following description below, it is to be understood that like numeric designations refer to components of like function.

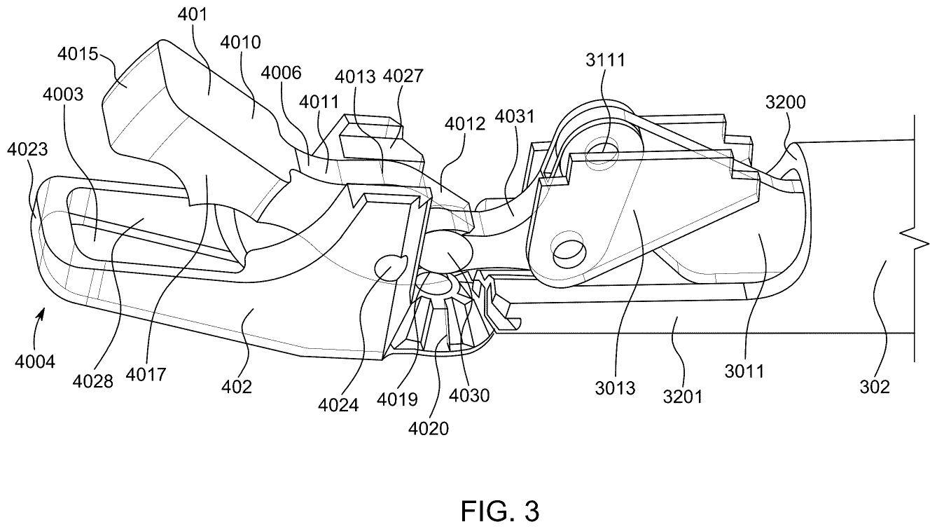

Turning now to FIGS. 1-3, there is illustrated an exemplary implementation of the surgical instrument having a swiveling end effector configured to deliver the same load at any yaw angle relative to a longitudinal axis of the surgical instrument. As illustrated, according to some embodiments, surgical instrument 10 may comprise a swiveling end effector 400 configured to swivel in a yaw direction of swivel shown by arrow 404, wherein swiveling end effector 400 may be operable to operate at constant load at any yaw swivel angle; and an elongated cylindrical shaft 300 defining longitudinal axis X_{L}, having a distal end operably coupled to swiveling end effector 400. The shaft 300 may have any suitable length depending on the relevant clinical application. For example, for a procedure to be performed on a knee the shaft length may be from 100-130 mm, for a procedure to be performed on a hip the shaft length may be from 150-220 mm, and for a procedure to be performed on a smaller joint the shaft length may be from 50-100 mm, according to some embodiments. The surgical instrument also includes a stationary handle 101, wherein a proximal end of the shaft 300 may be operably coupled to stationary handle 101; a stationary handle 101; an articulating handle 102, hingedly coupled to stationary handle 101 and operably coupled to first jaw 401 in swiveling end effector 400 the articulating handle 102 configured to operate swiveling end effector 400; and a swiveling member 200, operably coupled to second jaw 402 in swiveling end effector 400, operable to swivel swiveling end effector 400 to yaw swivel angle of ±120 degrees from a longitudinal axis X_{L}, wherein swiveling end effector 400 may be operable to transition between a closed position whereby first jaw 401 at least partially abuts second jaw 402, and an open position whereby the head portion distal end 4015 of the first jaw 401 may be at a distance from second jaw 402, as illustrated for example in FIG. 3, according to some embodiments.

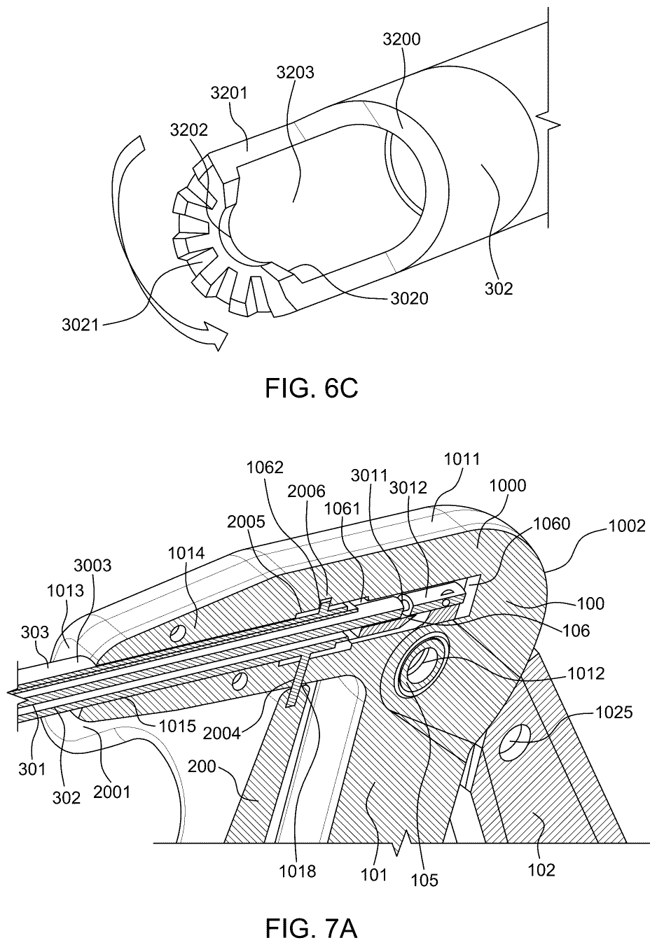

Turning now to FIGS. 3, 4A, 4B, 6A, and 6B there is illustrated the distal portion of a second jaw 402 (mandible) of an end effector, according to some embodiments, second jaw 402 having distal end 4004 and proximal end 4001, the end effector further comprising: a partially circular extension 4002 (see e.g., FIG. 3) extending proximally from proximal end 4001 of second jaw 402, partially circular extension 4002 having a basal surface 4005 and apical surface 4007, horizontal partial beveled gear 4021 extending apically from apical surface 4007 of partially circular extension 4002, horizontal partial beveled gear 4021 defining coaxial aperture 4020 extending through the basal surface 4005 of partially circular extension 4002, according to some embodiments. Second jaw 402 may further comprise annular, C-shaped front portion 4000, having external surface 4023 and internal surface 4028, defining an annulus wherein: a closed portion of C-shaped front portion 4000 defines the distal end of second jaw 402, apically flaring open portion 4026, 4026′, forming spilt shelf 4027 defining groove 4100 sized and configured to accommodate a portion of first jaw 401, with open portion 4026, 4026′ defining proximally beveled proximal end 4101, 4101′ (see e.g., FIG. 6B), sized, adapted and configured to provide yaw swivel freedom of ±120 degrees (in other words, a yaw angle of ±120°whereat, at a 0°position, end effector 400 front portion 4000 may be aligned with elongated cylindrical shaft 300 longitudinal axis XL), wherein a pair of coaxial horizontal bores 4024, 4024′ (FIG. 6B) are defined within apically flaring open portion 4026, 4026′ transverse to groove 4100 defined by split shelf 4027, according to some embodiments.

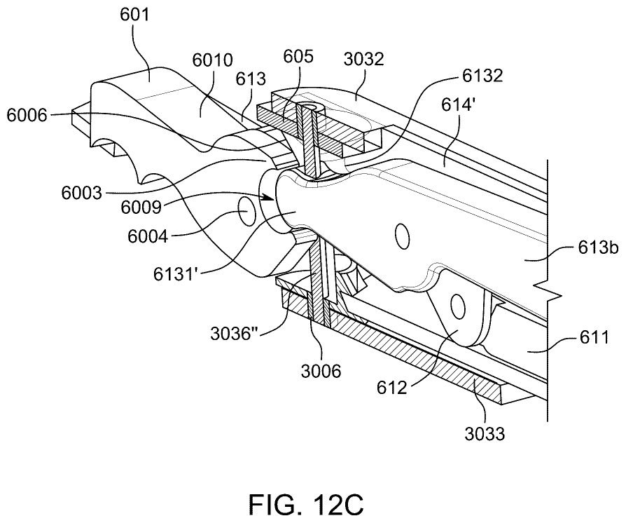

As further illustrated in FIGS. 6A and 6B, dorsal hinge member 405 (FIG. 4A), may be operably coupled apically to split shelf 4027, and has a portion 4050 extending proximally from split shelf 4027, defining an aperture 4051 therein (not shown, but similar or identical to aperture 6051 in dorsal hinge member 605 shown in FIG. 12A), aperture 4051 being coaxial with aperture 4020 defined by horizontal partial beveled gear 4021 coupled to apical surface 4007 of partially circular extension 4002, according to some embodiments.

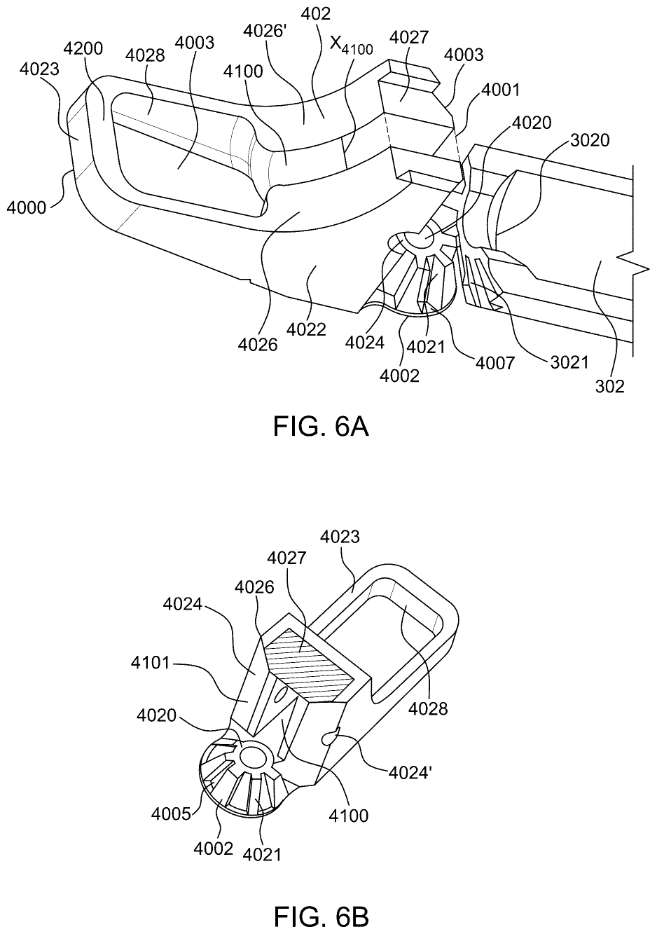

Turning now to FIGS. 3-5B, there is illustrated the distal portion of end effector 400 actuating mechanism for first jaw 401, according to some embodiments. As illustrated, first jaw 401 may include: head portion 4010 having distal end 4015 with potentially sharpened distal basal surface 4016, wherein head portion 4010 defines external peripheral surface 4017 (see e.g., FIG. 5A) that may be complimentary to internal surface 4028 defined by annulus 4003, and may be configured to be frictionally accommodated within annulus 4003 (see e.g., FIG. 3), according to some embodiments. Also illustrated is a lever 4006 having front section 4011, mid-section 4013, and rear section 4012 extending proximally from head portion 4010, front section 4011 and mid-section 4013 adapted, sized, and configured to be accommodated within groove 4100 defined by apically flaring open portion 4026, 4026′, wherein mid-section 4013 further defines through hole 4014 (see e.g., FIG. 5A), sized and configured to pivotally couple to apically flaring open portion 4026, 4026′ of second jaw 402, the lever 4006 having rear section 4012 defining a recess or circular depression 4019 having a contour that may be complimentary to distal portion of camming element 403, according to some embodiments.

As illustrated (see e.g., FIG. 4B), camming element 403, has a generally L-shape with a first leg 4031 having a ball joint 4030 disposed at a distal end of first leg 4031, while a proximal end of first leg 4031 may be coupled to second leg 4032 having a distal end defining through hole 4034, transverse to second leg 4032, and a proximal end terminating in pair of lateral extensions 4033, 4033′ forming gap 4037 therebetween, each lateral extension 4033, 4033′ defining pair of coaxial apertures 4035, 4035′ each disposed at a proximal end of one of the pair of lateral extensions 4033, 4033′, wherein a contour (see e.g., FIG. 5A) of circular depression 4019 defined in rear section 4012 of the lever 4006 extending proximally from head portion 4010 of first jaw 401 may be complimentary to OD₄₀₃₀ ball joint 4030, and may be configured to abut portion of ball joint 4030 or to have an effective contact area with ball joint 4030, according to some embodiments.

Alternatively, according to some embodiments, any suitably shaped component may be used at the distal portion of the camming element 403, such a component being complementary in shape and size to the depression in lever 4006, as discussed herein. Similar variations of sizes, shapes, and configurations of the components may be contemplated for any of the embodiments of the surgical instruments having swiveling end effectors discussed and illustrated herein.

According to some embodiments, instead of the camming element having a ball joint and the first jaw lever having a depression complementary to the ball joint, alternatively, the first jaw lever may be provided with a ball joint and the camming element may have a depression sized and shaped to be complementary to the ball joint on the first jaw lever. Similar variations of sizes, shapes, and configurations of the components may be contemplated for any of the embodiments of the surgical instruments having swiveling end effectors discussed and illustrated herein. Turning now to FIGS. 3, 4A, 5A, and 5B, there is illustrated the distal portion of end effector actuating mechanism for first jaw 401, according to some embodiments, which in the embodiment shown includes a pair of slider slabs 3013, 3013′. Slider slabs 3013, 3013′ are each substantially flat members sized and configured to straddle on their broad side portions of lever front section 4011 and midsection 4013, according to some embodiments. Each slider slab 4013 (4013′) has a narrow distal facet 3019d (3019d') that may be larger than opposing narrow proximal facet 3019p (3019p′)′ (see e.g., FIG. 5B), with a slider slab aperture 3014 (3014′) defined through slider slab 3013 (3013) toward a basal portion of the slider slab and towards distal facet 3019d (3019d′), according to some embodiments. A tab 3015 (3015′) extends between distal facet 3019d (3019d′) and proximal facet 3019p (3019p′) of slider slab 3013 (3013′), tab 3015 (3015′) having a distal wall 3016 (3016′) and a proximal wall 3017 (3017′), according to some embodiments.

Also illustrated in FIG. 5A is first hinging element 3018, pivotally coupling the pair of slider slabs 3013, 3013′ to camming element 403 via through hole 4034 (see e.g., FIG. 4B) defined in distal end of second leg 4032 of L-shape camming element 403, according to some embodiments. Likewise second hinging element 4029 pivotally couples second jaw 402 and first jaw 401 by extending through hole 4014 defined in mid-section 4013 of lever 4006 extending proximally from head portion 4010 of first jaw 401, disposed within groove 4100 defined by split shelf 4027, through hole 4014 being coaxial with the pair of coaxial horizontal bores 4024, 4024′ defined within the apically flaring open portion 4026, 4026′ transverse to groove 4100, according to some embodiments.

Further and as illustrated in FIGS. 3-4B, swiveling end effector 400 may further include: dorsal coupling member 3032, coupled to and extending distally from distal end 3031 (see e.g., FIG. 4A) of elongated cylindrical shaft 300, according to some embodiments. Dorsal coupling member 3032 may include: a distal portion 3008 (FIG. 12A), defining dorsal aperture 3034, dorsal aperture 3034 being coaxial with aperture 4050 defined in dorsal hinge member 405; and a proximal portion 3007, defining rectangular window 3035, rectangular window's 3035 narrow aspect sized and configured to accommodate lateral extensions 4033, 4033′ of second leg 4032 of camming element 403, sandwiched between tabs 3015, 3015′ extending apically from respective slider slabs 3013, 3013′, according to some embodiments. Also shown (FIG. 12A) is ventral coupling member 3033, coupled to and having distal portion 3006 extending distally from distal end 3031 of elongated cylindrical shaft 300, distal portion 3006 of ventral coupling member 3033 further defining ventral aperture 3005 which may be coaxial with coaxial aperture 4020 (see e.g., FIG. 6B) of horizontal partial beveled gear 4021, extending through basal surface 4005 of partially circular extension 4002 of second jaw 402; and third hinging element 3036, pivotally coupling dorsal hinge member 405, via aperture 4050 therein, to split shelf 4027, and distal portion 3008 of dorsal coupling member 3032 via aperture 3034 ; and fourth hinging element 3037, pivotally coupling ventral coupling member 3033, via aperture 3005, and basal surface 4005 of partially circular extension 4002 of second jaw 402, via coaxial aperture 4020, according to some embodiments.

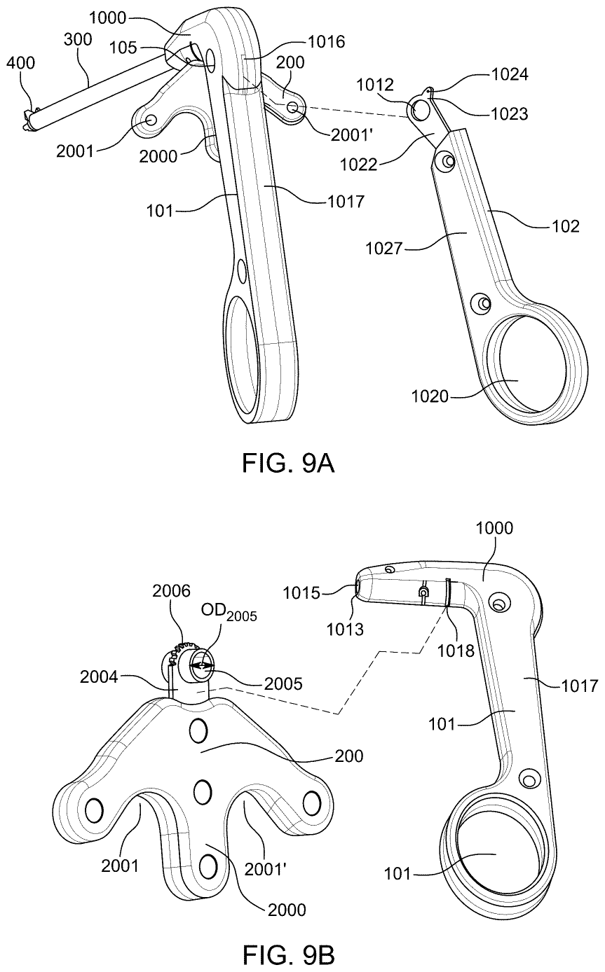

Turning now to FIGS. 1, 2, 5A, 6C, 7A, 7B, and 8, there is illustrated elongated cylindrical shaft 300 comprising sleeve 303 (see e.g., FIG. 8), shaft 300 having proximal end 3003 (see e.g., FIG. 7A) coupled to stationary handle 101, and distal end 3031 coupled to dorsal coupling member 3032 and ventral coupling member 3033 (FIG. 4A), according to some embodiments. Elongated cylindrical shaft 300 may further include elongated cannula 302 nested within, and coaxial with sleeve 303, cannula 302 having proximal end 3023 operably coupled to swiveling member 200, and distal end 3200 (see e.g., FIG. 6C) having partially cylindrical extension 3201 defining vertical partial beveled gear 3021 at its distal end, vertical partial bevel gear 3201 configured to pivotally engage horizontal partial beveled gear 4021, such that rotating elongated cannula 302 using swiveling member 200 will cause end effector 400 to swivel about swivel axis (yaw axis) X_{Y} (see e.g., FIG. 4A), according to some embodiments.

As further illustrated in FIGS. 5A, 7A, 7B and 8, elongated cylindrical shaft 300 may further include rod assembly 301, nested within elongated cannula 302 (see e.g., FIG. 8), rod assembly 301 having proximal end 3100 (see e.g., FIGS. 7A, 7B) operably coupled to articulating handle 102, and distal end 3010 (see e.g., FIG. 5A) pivotally coupled to camming element 403, according to some embodiments. As further illustrated in FIG. 7A, and 7C, stationary handle 101 may further include head portion 1000 having distal end 1013 and proximal end 1002, and body portion 1017, wherein head portion 1000 defines shaft bore 1015 coaxial with longitudinal axis X_{L} of elongated cylindrical shaft 300, shaft bore 1015 having distal end 3002 and proximal end 3001, according to some embodiments. As illustrated in FIG. 7C, head portion 1000 further defines first cavity 106 expanding from proximal end 3001 of shaft bore 1015, first cavity 106 adapted, sized and configured to accommodate a portion of swiveling member 200; and second cavity 1060, adapted sized and configured to accommodate a portion of articulating handle 102 and a portion of rod assembly 301, according to some embodiments.

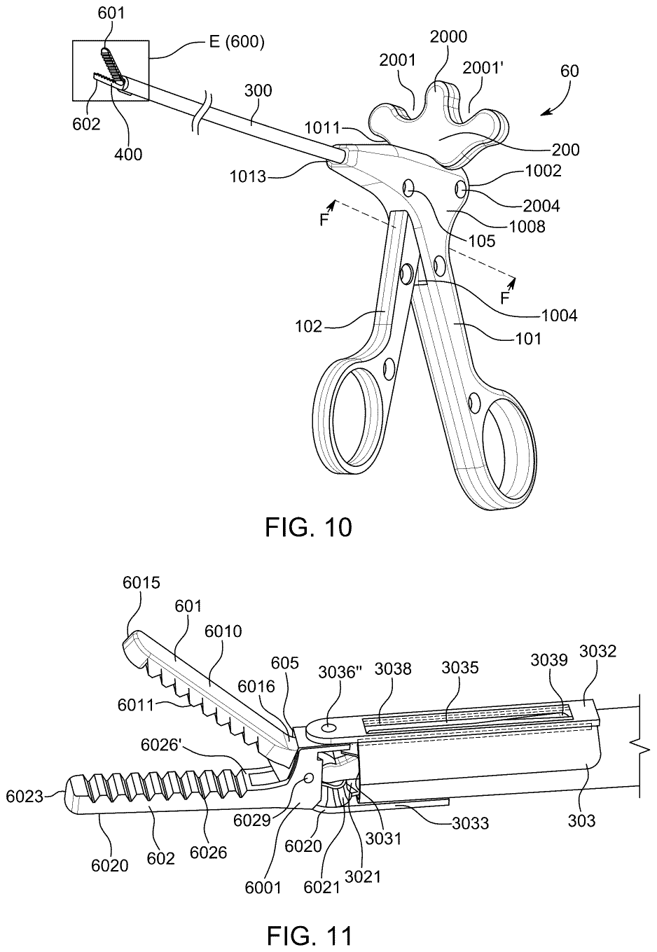

Further, and as illustrated in FIGS. 7A, 7C, 9A, and 9B head portion 1000 further defines: a hinge aperture 1012 configured to receive sixth hinging element 105 (see e.g. FIG. 7A—a bobbin hinge), with proximal slot 1016 extending through proximal end 1002 of head portion 1000, through hinge aperture 1012, to second cavity 1060; and partially circular ventral slot 1018 (see e.g., FIG. 9B), extending apically to first cavity 106, according to some embodiments.

Turning now to FIGS. 7A, 7B, and 9A, there is illustrated articulating handle 102 as further comprising linking member 1022, extending apically from distal end 1021 of articulating handle 102, linking member 1022 sized and configured to be accommodated in proximal slot 1016 of head portion 1000 of stationary handle 101, wherein linking member 1022 further defines apical protrusion 1023 (see e.g., FIG. 9A), operable to pivotally couple to proximal member 3012 (inside second cavity 1060), and wherein linking member 1022 may be hingedly coupled to stationary handle 101 via sixth hinging element 105, according to some embodiments.

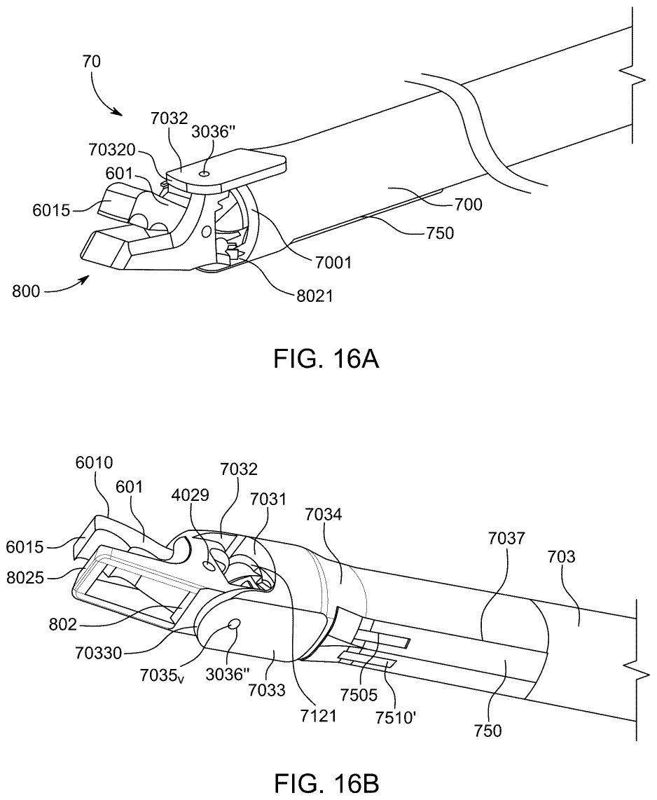

With reference to FIGS. 3, 4A, 4B, 5A, 7A, and 7B, rod assembly 301 may include: push-rod 3101 (FIG. 7A) having proximal end 3102 and distal end 3010 (FIG. 16D), with proximal member 3012 (FIG. 7B) extending proximally from proximal end 3102 of push rod 3101 (see e.g., FIG. 7B); and camming linker 3011 (FIG. 3), extending diagonally, or apically and distally at a predetermined angle from distal end 3010 of push rod 3101 (see e.g., FIG. 5A), according to some embodiments. Camming linker 3011 may be sized to be accommodated within gap 4037 (d₄₀₃₇) formed between lateral extensions 4033, 4033′ of camming element 403, and may be operable to hingedly couple to camming element 403 via fifth hinging element 4036 extending through coaxial apertures 4035, 4035′ in lateral extensions 4033, 4033′ and an aperture 3111 in camming linker 3011, according to some embodiments.

Reference is now made to FIGS. 1, 2, 6C, 7A-C, and 9B in relation to swiveling element 200. As illustrated, swiveling member 200, according to some embodiments, may include: basal body portion 2000 defining right depression 2001 and left depression 2001′ (for accommodating a right-handed or left handed clinician where the dominant index finger location relative to the stationary handle 101 will vary), with flat neck portion 2004, sized and configured to be accommodated within partially circular ventral slot 1018 (see e.g., FIG. 9B) of head portion 1000 of stationary handle 101, according to some embodiments. Additionally, a hollow cylinder 2005 extends transverse to (crossing) neck portion 2004, wherein hollow cylinder 2005 may be configured to couple to proximal end 3023 of elongated cannula 302; and an arcuate indexing teeth portion 2006 (also referred to as “ratchet teeth 2006”) may be disposed at the end of flat neck 2004 portion and transverse to longitudinal axis X_{L2005} defined by hollow cylinder 2005, according to some embodiments. Correspondingly, and as illustrated in FIG. 7C, first cavity 106 further defines radial channel 1062 configured to accommodate ratchet teeth 2006, first cavity 106 further comprising resilient tab 1065, disposed transverse to-, and within, radial channel 1062, resilient tab 1065 (acting as a ratchet pawl) sized, adapted, and configured to releasably engage ratchet teeth 2006, according to some embodiments. In the context of the disclosure, the term “swiveling member” means a member incorporating means for torque-transferring engagement with the elongated cannula 302 and the first cavity 106, and such engagement means may but will not necessarily comprise ratchet teeth 2006 as such, according to some embodiments. The skilled artisan will recognize that alternative torque-transfer engagement means may be devised using known technologies without departing from the scope of the disclosed swivel indexing mechanism. The number of ratchet teeth may be predetermined in an exemplary implementation to provide fixed angle periodicity to the yaw swivel angle. Head portion may further include distal portion 1014

In an exemplary implementation, the instruments disclosed are used in various surgical procedures, such as for example meniscectomy. Accordingly and as illustrated in the figures, biasing articulating handle 102 toward stationary handle 101, will cause apical protrusion 1023 disposed on linking member 1022 to rotate clock wise, pulling proximal member 3012 proximally, which will cause camming linker 3011 to pull camming element 403 proximally, according to some embodiments. Since slider slabs 3013, 3013′ are hingedly coupled to camming linker 3011 by first hinging element 3018, slider slabs 3013, 3013′ will likewise retract proximally within rectangular window 3035 (see e.g., FIG. 4A), defined in dorsal coupling member 3032, causing proximal walls 3017 and 3017′ of tabs 3015 and 3015′ on slider slabs 3013 and 3013′ to abut proximal facet 3039 of rectangular window 3035 (3038 being the distal facet), and since as indicated slider slabs 3013, 3013′ are hingedly coupled to camming element 403 by first hinging element 3018, via through hole 4034, first leg 4031 of camming element 403 will also rotate clock-wise, causing ball joint 4030 to lift rear portion 4012 of the lever 4006 to rotate counter clock-wise causing head portion 4010 to be accommodated and frictionally engaging annulus 4003, according to some embodiments. As needed, and using articulating eyelet 1020 and stationary eyelet 1010 (if present), using the reverse process, a clinician can expand a space for accessing a surgical site, according to some embodiments. In an exemplary implementation, swiveling end effector 400 may be configured to provide a compression load of between about 29.42 newton and about 98.00 newton at any yaw swivel angle between first jaw 401 and second jaw 402, according to some embodiments. It is noted that swivel axis (yaw axis) X_{Y} formed by third hinging element 3036, fourth hinging element 3037 and the center of ball joint 4030, all which are coaxial, allows the instrument to generate torque of between about 150 N·mm and about 600 N·mm on the jaws such as, for example, from about 150-250 N-mm, from about 150-300 N-mm, from about 300-400 N-mm, from about 350-450 N-mm, or from about 400-600 N-mm,, at a shaft diameter that may be less than 4.5 mm such as, for example, 4 mm or 3.5 mm or 3 mm or 2.5 mm or 2 mm, at any yaw articulation angle, according to some embodiments.

According to some embodiments, for a shaft diameter of 4.0-4.5 mm the surgical instrument may generate a torque of from 400-600 N-mm.

According to some embodiments, for a shaft diameter of 3.5-4.0 mm the surgical instrument may generate a torque of from 350-450 N-mm According to some embodiments, for a shaft diameter of 3.0-3.5 mm the surgical instrument may generate a torque of from 300-400 N-mm.

According to some embodiments, for a shaft diameter of 2.5-3.0 mm the surgical instrument may generate torque of less than 300 N-mm. It may be noted that a surgical instrument generating a torque smaller than 300 N-mm may not be able to cut a meniscus.

According to some embodiments, for a shaft diameter of 2.0-2.5 mm the surgical instrument may generate a torque of less than 250 N-mm. It may be noted that a surgical instrument generating a torque smaller than 250 N-mm may be possible with a much smaller tip size.

For example, for a surgical instrument having an end effector including a biting tool with a length of 5.5 mm and a shaft having an outer diameter of 4.2 mm, the surgical instrument is configured to generate torque of about 525 N-mm at a distal portion of the biting tool.

Simultaneously (or not), end effector 400 can be swiveled (yawed) relative to elongated cylindrical shaft 300 longitudinal axis X_{L}, by tilting swiveling member 200 either clockwise, or counter-clockwise in partially circular ventral slot 1018, imparting torque on elongated cannula 302 to rotate radially within sleeve 303 relative to elongated cylindrical shaft 300 longitudinal axis X_{L}, with ratchet teeth 2006 releasably engaging resilient tab 1065 (acting as a ratchet pawl) while at the distal end 3200 of elongated cannula 302, partially cylindrical extension 3203 having at its distal end vertical partial beveled gear 3021, will engage horizontal partial beveled gear 4021 extending apically from apical surface 4007of partially circular extension 4002, and convert the radial rotation about elongated cylindrical shaft 300 longitudinal axis X_{L}, to rotation about swivel axis Xy formed by third hinging element 3036, fourth hinging element 3037 and the center of ball joint 4030, all which are coaxial, according to some embodiments. It may be noted that in an exemplary implementation, the horizontal and vertical partial beveled gears may be replaced with horizontal and vertical partial pinwheel gears, or a circular rack-and-pinion (face gear and pinion), according to some embodiments. In an exemplary implementation, and to impart on swiveling member 200 full range of motion, partially circular ventral slot 1018 extends ±2.09 rad. (or ±120°) off vertical, according to some embodiments.

It may be noted that, optionally, the degree of tilting of swiveling member 200, as discussed herein, may be directly proportional or indirectly proportional to the degree of rotation of the end effector about swivel axis Xy, according to some embodiments. For example, tilting of swiveling member 200 by 90 degrees may cause a corresponding swiveling of end effector by 90 degrees, according to some embodiments. Alternatively, according to some embodiments, there may be an indirectly proportional correspondence between tilting of swiveling member 200 and swiveling of the end effector such as, for example, a 5:6 ratio, or a 6:7 ratio, or a 7:8 ratio, or any other selected ratio, depending on selected gear ratios, according to some embodiments.

Moreover, in certain exemplary implementations, surgical instrument 10 may further include a powertrain (in other words motors and gears) operably coupled to at least one of the swiveling member 200, the articulating handle 102, and the stationary handle 101, for example, a first powertrain (not shown) coupled to the swiveling member 200, operable to swivel the swiveling end effector; and a second powertrain, operable to transition the first jaw 401 from an open position to a closed position, according to some embodiments.

In addition, and as illustrated, hollow cylinder 2005 of swiveling member 200 may have the same outer diameter as elongated cylindrical shaft 300 [OD₂₀₀₅=OD₃₀₀], as shown, e.g., in FIG. 8, and 9B, according to some embodiments. For example, the outer diameter of shaft 300 may be have an outer diameter of from 2-4.5 mm such as, for example, from 2-3 mm, from 2.5-3.5 mm, or from 3.5-4.5 mm.

In addition and in certain exemplary implementations, annular, C-shaped front portion 4000 defines a rectangular annulus 4003 whereby long facets of the rectangular annulus 4003 are parallel to longitudinal axis XL, of elongated cylindrical shaft 300, wherein: short facets of the rectangular annulus 4003 have a dimension of between 1.0 mm and 5.0 mm; the long facets of rectangular annulus 4003 have a dimension of between 2.0 mm and 6.0 mm; and in the open position of the end effector 400, the distance between the distal basal surface 4016 of head portion 4010 of first jaw 401 and upper surface 4200 of annulus 4003 front portion 4000 may be between 1.5 mm and 6.0 mm, according to some embodiments.

Furthermore, in certain exemplary implementations, first jaw 401, and second jaw 402 are formed of dielectric material, for example, ceramic, zirconia, quartz and the like, wherein first jaw 401 and second jaw 402 further comprise respective conductive elements(not shown) and wherein surgical instrument 10 may further include an anode coupled to first jaw 401 and cathode coupled to second jaw 402 or vice versa, in other words, an anode coupled to second jaw 402 and cathode coupled to first jaw 401, according to some embodiments.

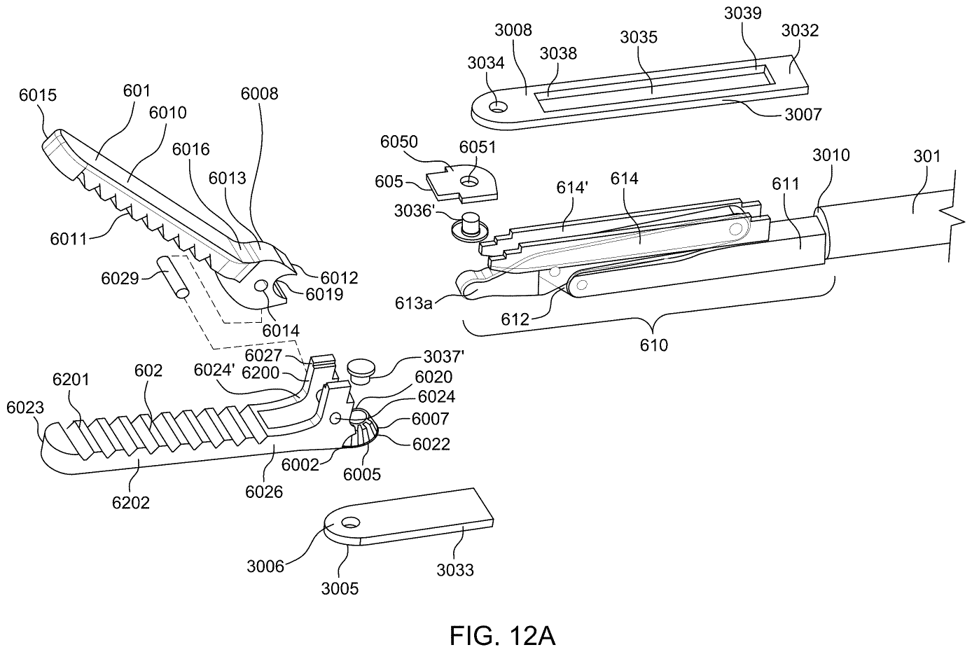

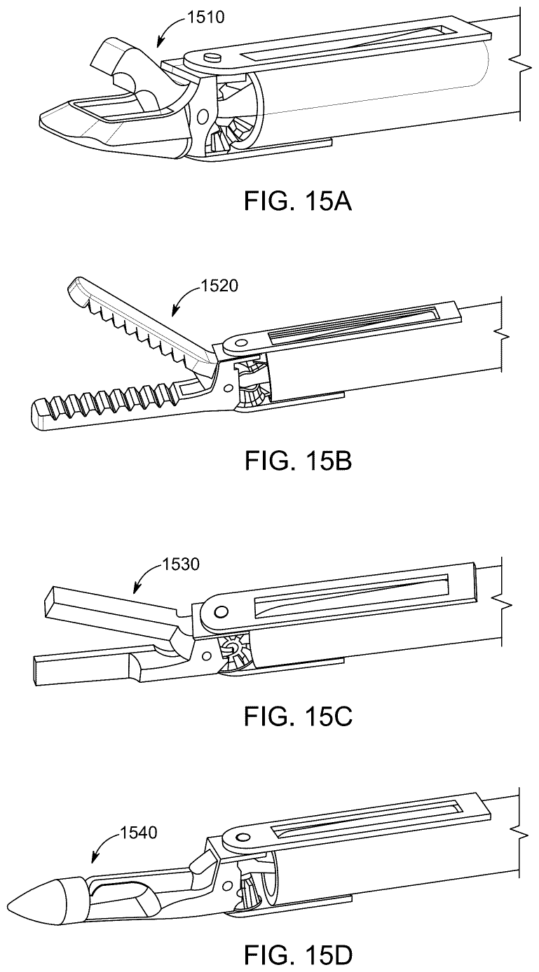

It may be noted that the shape of the jaws 401 and 402, in particular the second (lower) jaw 402, may facilitate interaction between the jaws of the end effector and tissue (not shown) to which the end effector 400 is applied. For example, an end effector having an angled portion on the lower jaw, such as, for example shown in FIG. 15A, may potentially allow the lower jaw to be more easily inserted beneath tissue such as, for example, a meniscus, thereby allowing better positioning of the jaws relative to the tissue. Other potential advantages of end effectors having portions that are thinner such as, for example, as shown in FIG. 15C; or having ridges such as, for example, as shown in FIG. 15B; or pointed such as for example, as shown in FIG. 15D will be understood by persons skilled in the art. Another exemplary implementation of the surgical instrument comprising a swiveling end effector operable to deliver a predetermined torque independent of a yaw swivel angle is illustrated in FIGS. 10-14B, according to some embodiments. FIG. 10 illustrates a surgical instrument 60 comprising a swiveling end effector 600 operable to deliver a predetermined torque independent of a yaw swivel angle, as discussed herein. In the context of the disclosure, the predetermined torque refers to the force at which the surgical instrument's jaws close, according to some embodiments. As indicated, the end effector can be a punch 1510 (see e.g., FIG. 15A), a grasper 1520 (see e.g., FIG. 15B), bypass scissors 1530 (see e.g., FIG. 15C), a suture passer 1540 (see e.g., FIG. 15D), or anvil scissors (not shown), according to some embodiments.

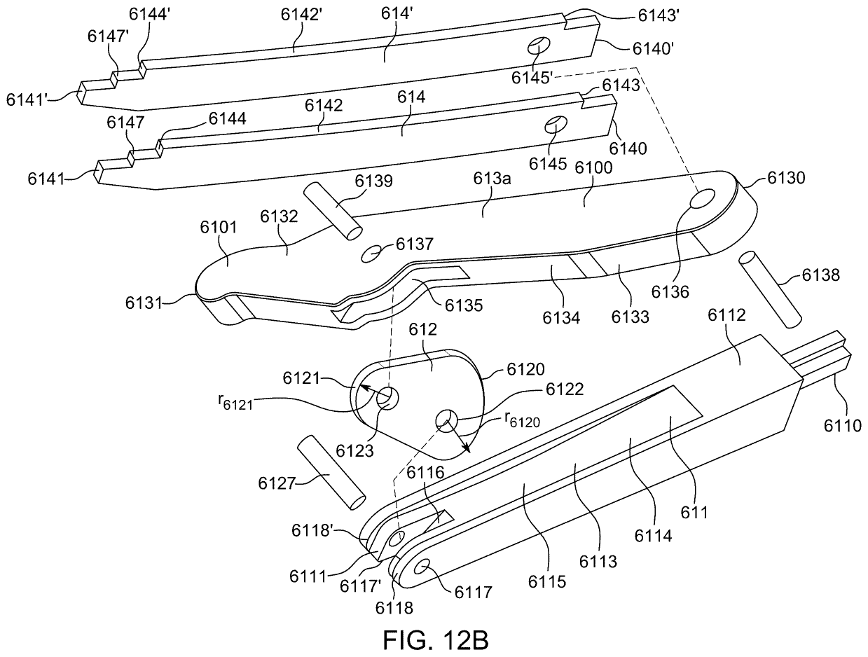

As illustrated in FIG. 10, and different from FIG. 1, the articulating handle 102 may be distal to stationary handle 101, according to some embodiments. As illustrated surgical instrument 60 may include: swiveling end effector 600 (grasper) operable to deliver a predetermined torque independent of a yaw swivel angle; elongated cylindrical shaft 300 defining longitudinal axis XL (see e.g., FIG. 1), having distal end operably coupled to swiveling end effector 600, and proximal end operably coupled to stationary handle 101; stationary handle 101; articulating handle 102, hingedly coupled to stationary handle 101 and operably coupled to first jaw 601 in swiveling end effector end 600, configured to operate swiveling end effector 600; and swiveling member 200, operably coupled to second jaw 602 in swiveling end effector 600, operable to swivel swiveling end effector 600 to yaw swivel angle of ±120 degrees from longitudinal axis X_{L} (see e.g., FIG. 1), wherein swiveling end effector 600 may be operable to transition between a closed position whereby first jaw 601 at least partially abuts second jaw 602, and an open position whereby the head portion distal end 6015 of first jaw 601 may be at a distance from second jaw 602, as illustrated for example in FIG. 11, according to some embodiments.

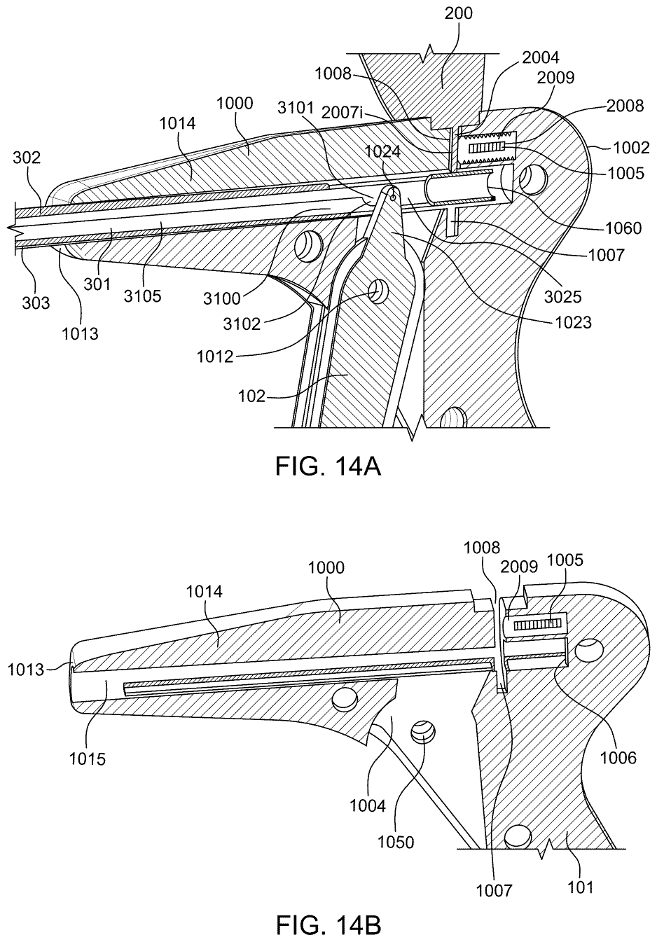

While some embodiments are discussed with relation to articulating handle 102 being proximal to stationary handle 101 such as, for example, that shown in FIG. 7A, it will be appreciate by persons skilled in the art that, alternatively, any of these embodiments may be provided with an articulating handle 102 being distal to stationary handle 101 such as, for example, in the embodiment shown in FIG. 14A.

Turning now to FIGS. 11 and 12A, there is shown the distal portion of end effector 600 actuating mechanism for first jaw 601 (mandible),, according to some embodiments, the second jaw having distal end 6023 and proximal end 6022, the actuating mechanism further comprising: partially circular extension 6002 (see e.g., FIG. 12A) extending proximally from second jaw proximal end 6001, partially circular extension 6002 having basal surface 6005 and apical surface 6007, horizontal partial beveled gear 6021 extending apically from apical surface 6007 of partially circular extension 6002, horizontal partial beveled gear 6021 defining coaxial aperture 6020 extending through the basal surface 6005 of partially circular extension 6002, according to some embodiments. Second jaw 602 may further include serrated surface 6201, and basal surface 6202, apically flaring open portion 6026, 6026′, forming spilt shelf 6027 (see e.g., FIG. 12A) defining groove 6200 sized and configured to accommodate a portion of first jaw 601, wherein pair of coaxial horizontal bores 6024, 6024′ are defined within apically flaring open portion 6026, 6026′ transverse to groove 6200 defined by split shelf 6027, according to some embodiments.

As further illustrated in FIG. 11, and 12A, dorsal hinge member 605, operably coupled apically to split shelf 6027, having portion 6050 extending proximally from split shelf 6027, defining aperture 6051 therein, aperture 6051 being coaxial with aperture 6020 defined by horizontal partial beveled gear 6021 coupled to apical surface 6007 of partially circular extension 6002, according to some embodiments.

As further illustrated (see e.g., FIG. 12A), first jaw 601 may include: head portion 6010 having distal end 6015 with potentially serrated basal surface 6011, according to some embodiments. Also illustrated is lever 6008 having front section 6016, mid-section 6013, and rear section 6012 extending proximally from head portion 6010, according to some embodiments. Front section 6016 and mid-section 6013 are adapted, sized, and configured to be accommodated within groove 6200 defined by apically flaring open portions 6026, 6026′, wherein mid-section 6013 further defines through hole 6014 (see e.g., FIG. 12A), sized and configured to pivotally couple to apically flaring open portions 6026, 6026′ of second jaw 602, the lever 6008 having rear section 6012 defining a recess or circular depression 6019 having contour that may be complimentary to a distal portion of a camming arm 613 (613a in FIGS. 12A-B; 613b in FIG. 12C), according to some embodiments. Two examples of camming arm 613 are illustrated and described herein, namely camming arms 613a and 613b. It may be to be understood that these examples are exemplary only, and it will be appreciated by persons skilled in the art that, alternatively, a camming arm having another structure may be utilized for a similar or equivalent function, as discussed herein.

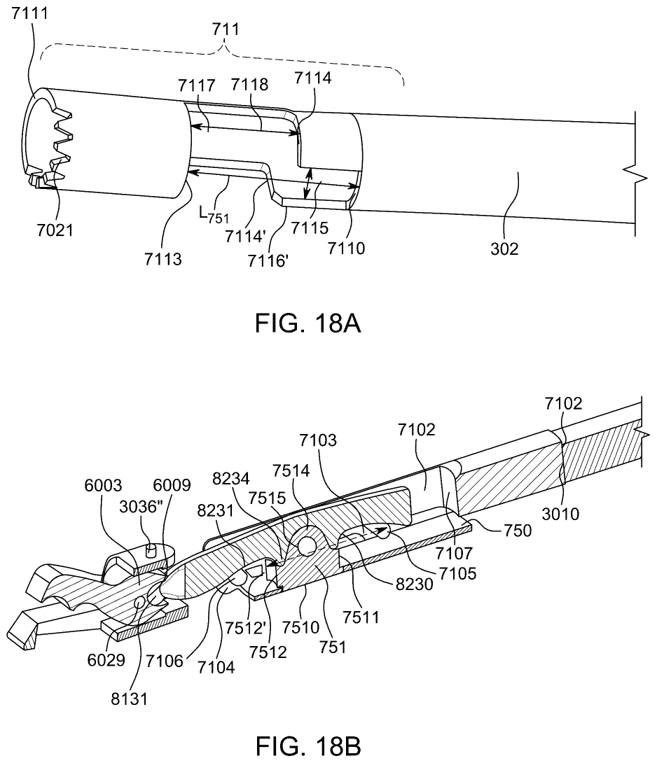

It will be appreciated by persons skilled in the art that, instead of having a portion of the first jaw including a depression or recess that may be complimentary to and mate with a portion on a camming element (for example, ball joint 4030 or on camming element 403, shown in FIG. 4B) or a camming arm (cylindrical distal portion of camming arm 613b, shown in FIG. 12C) or on a camming element (for example, frusto-spherical distal end 8131 of camming element 813 in FIG. 19), alternatively, the camming element/camming arm may include a depression or recess that mates with a cylindrical or spherical or frusto-spherical component on the first jaw, whereby force is applied to the handle(s) in order to transmit torque to the upper jaw, as discussed herein.

It may be noted that a torque ratio between the articulating handle 102 and the first jaw 401 may be, for example, about 1:2.5, according to some embodiments. In other words, for a torque of about 38 N applied at the articulating handle 102, a torque of about 95 N may be applied at the first jaw 401.

According to some embodiments, when it is desired to open the jaws, a force of about 25-50 N-mm applied to the articulating handle 102 such as, for example, from about 25-35 N-mm, or from about 30-40 N-mm, or from about 40-50 N-mm, may result in a torque applied to the first jaw 401 to open the first jaw relative to the second jaw 402, the torque being from about 60-90 N-mm, or from about 75-100 N-mm, or from about 100-125 N-mm, according to some embodiments.

Optionally, the articulating handle 102 may be spring-loaded, such that it will return to the position it was in prior to being moved to close the first jaw 401 relative to the second jaw 402, according to some embodiments. Optionally, the articulating handle 102 has an associated ratchet mechanism, such that it may be effectively locked in positioned after the first jaw 401 is closed relative to the second jaw 402. This may be important, for example, if the end effector includes a grasping tool.

Turning now to FIGS. 11-12B, there is illustrated an end effector power-amplifying actuating assembly for first jaw 601, comprising: elongated slider 611 having proximal end 6110 coupled to distal end of rod assembly 301, and distal end 6111, elongated slider 611 further having upper surface 6113 defining channel having sloped portion 6114 and horizontal portion 6115, upper surface 6113 configured to partially accommodate complementary basal surfaces 6133, 6134 of camming arm 613a, wherein distal end 6111 further includes pair of extensions 6118, 6118′ extending distally, defining axial groove 6116 adapted, sized, and configured to partially accommodate coupling slab 612, extensions 6118, 6118′ further defining respective coaxial horizontal bores 6117, 6117′ transverse to axial groove 6116, according to some embodiments. It may be noted that, in this embodiment (FIG. 12A-B), the distal portion of the camming arm 613a is shown as having a cylindrical, semicircular profile. Lever 6008 of first jaw 601 includes a depression 6019 that may be sized and shaped to correspond to the distal portion of camming arm 613a, as seen most clearly in FIG. 12A, according to some embodiments. It will be appreciated by person skilled in the art that, alternatively, any suitably shaped component may be used at the distal portion of the camming arm 613a, such a component being complementary in shape and size to the depression in lever 6008, as discussed herein, according to some embodiments.

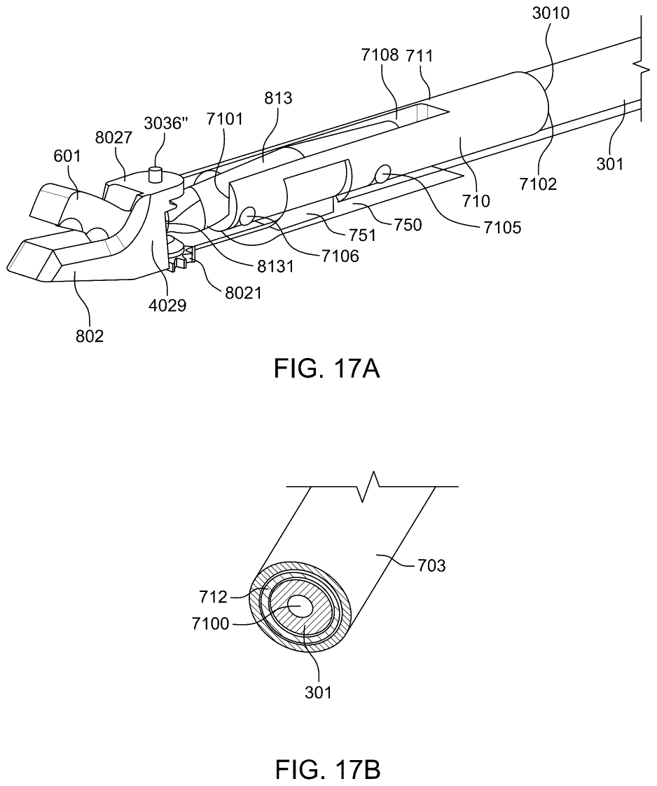

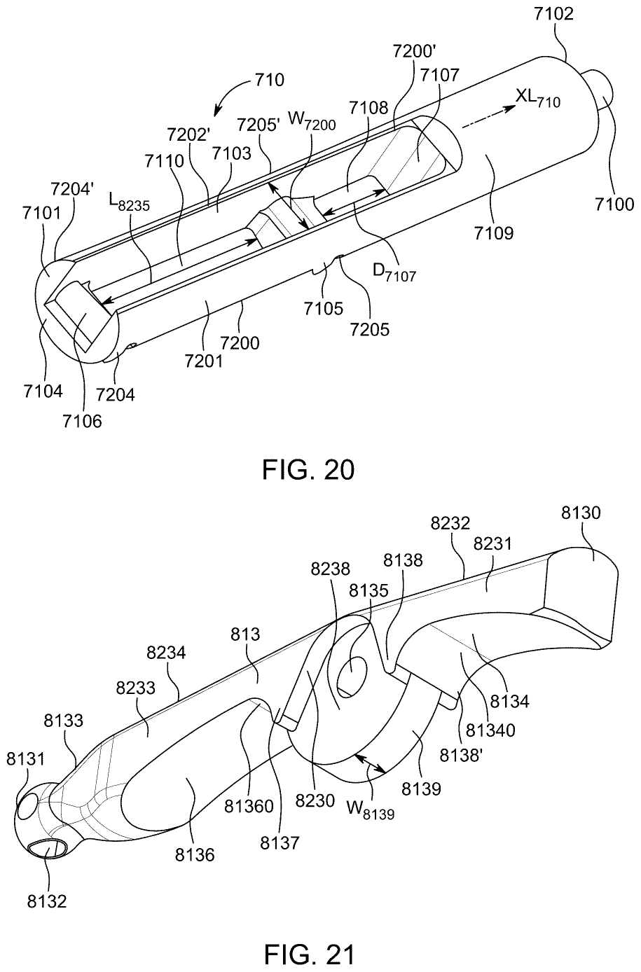

In an alternative embodiment, the distal end portion of the camming arm (813 in FIG. 21) may have an alternative configuration such as, for example, frusto-spherical, and the corresponding depression in the first jaw (depression 6019 in FIG. 12A) may be sized and shaped to be complimentary to the frusto-spherical (or other-shaped) distal end portion of this camming arm. It will be appreciated by person skilled in the art that, alternatively, any suitably shaped component may be used at the distal portion of the camming arm 813, such a component being complementary in shape and size to the depression 6009 in the first jaw lever (FIG. 18B), as discussed herein.

It may be noted that some components of the surgical instrument 60, including camming arm 613a (or camming arm 813 in FIG. 21) may be identical or similar in structure and function to those of surgical instrument 10 (FIG. 1), and may not be described again herein.

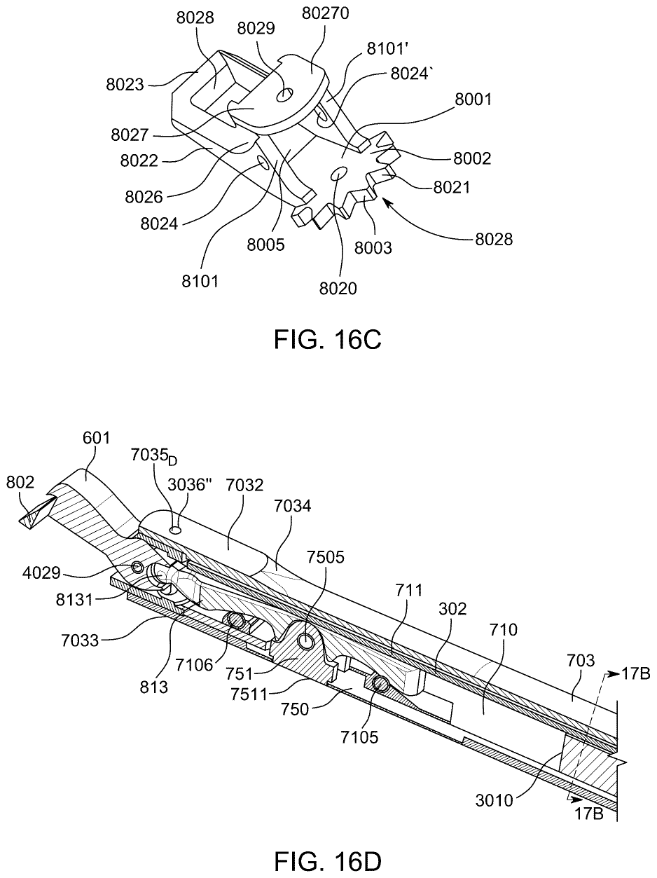

Turning now to FIG. 12C, there is illustrated an alternative swiveling assembly having a single swiveling pin, (aka, third hinging element 3036″, see e.g. FIGS. 12C and 17A) and a camming arm 613b having a split/perforated distal end, according to some embodiments. As illustrated, first jaw rear section 6012 (see e.g., FIG. 12C) may be comprised of two lobes 6003, 6003′ (the latter lobe 6003′, not shown, symmetric to lobe 6003), the lobes defining a pair of respective through holes 6004, 6004′ and respective circular depressions 6009, 6009′ in the lobes 6003, 6003′ forming a groove 6006 between lobes 6003, 6003′, groove 6006 sized and configured to accommodate third hinging element 3036″, according to some embodiments. Here too, circular depressions 6009, 6009′ may each have a contour that may be complimentary to distal end portions 6131, 6131′ of camming arm 613b. It may be noted that since the distal end of camming arm 613b may be split into portions 6131 and 6131′, as discussed herein, there may be formed a channel 6132 between portions 6131 and 6131′, the channel 6132 configured to accommodate a third hinging element 3036″ (in other words, swiveling pin shown in FIG. 11), according to some embodiments. It may be noted that, in this embodiment, due to the channel 6132 between portions 6131, 6131′ in camming arm 613b, the third hinging element 3036″ may provide the functionality of the third hinging member 3036′ and the fourth hinging member 3037′ in the embodiment of FIG. 12A. It will be appreciated by person skilled in the art that, alternatively, any suitably shaped component may be used at the distal portion of the camming arm 613b, such a component being complementary in shape and size to the depression 6009 in the first jaw lever, as discussed herein.

As will be illustrated herein, first jaw 601, can be used in conjunction with second jaw 802 and alternative camming arm 813 (see e.g., FIGS. 16A-C, 18A-19, and 21), according to some embodiments, and as discussed further herein.

Further, coupling slab 612 has an arcuate distal end 6121 and an arcuate proximal end 6120, wherein a diameter r₆₁₂₀ defined by arcuate proximal end 6120 is larger than a diameter r₆₁₂₁ defined by arcuate proximal end 6121, according to some embodiments. Coupling slab 612 may define distal through hole 6123 and proximal through hole 6122.

Similarly, camming arm 613 may have proximal end 6130 and distal end 6131, distal portion 6101 and proximal portion 6100, wherein distal portion 6101 defines a cavity 6135 configured to accommodate arcuate distal end 6121 of coupling slab 612, according to some embodiments. Distal portion 6101 further defines pair of apertures 6137, 6137′ (the latter aperture not shown) sized, adapted and configured coaxially with distal through hole 6123 defined in arcuate distal end 6121 of coupling slab 612, and wherein proximal portion 6100 may further include a drill hole 6136 defined towards proximal end 6130, according to some embodiments.

In addition, as illustrated in FIG. 12B, end effector 600 power-amplifying actuating assembly may further include a pair of slider brackets or bracket slabs 614, 614′, having respective narrow distal facets 6141, 6141′ and respective opposing narrow proximal facets 6140, 6140′, with bracket slab apertures 6145, 6145′ defined through respective bracket slabs 614, 614′ in proximity to proximal ends (proximal facets) 6140, 6140′ of slider bracket 614, 614′, according to some embodiments. Tabs 6142, 6142′may extend apically from respective upper narrow facets 6147, 6147′ of respective bracket slabs 614, 614′, tabs 6142, 6142′ having respective distal walls 6144, 6144′ and proximal walls 6143, 6143′, according to some embodiments.

End effector 600 power-amplifying actuating assembly further utilizes a seventh hinging element 6138, which may pivotally couple the pair of slider brackets 614, 614′ to drill hole 6136 defined in camming arm proximal end 6130, by extending through bracket slab apertures 6145, 6145′, according to some embodiments. Also eighth hinging element 6029 (FIG. 11), may pivotally couple second jaw 602 to first jaw 601 by extending through hole 6014 (FIG. 12A) defined in mid-section 6013 of lever 6008, the lever 6008 disposed within groove 6200 defined by split shelf 6027, and the eighth hinging element 6029 extending through a pair of coaxial horizontal bores 6024, 6024′ defined within apically flaring open portion 6026, 6026′ transverse to groove 6200, according to some embodiments. A ninth hinging element 6139 may pivotally couple slab 612 and camming arm 613, by extending through a pair of apertures 6137, 6137′ (the latter of which is not shown) defined in distal portion 6101 of camming arm 613 and coaxial with distal through hole 6123 defined in coupling slab arcuate distal end 6121, the coupling slab 612 being accommodated in camming arm cavity 6135, according to some embodiments. A tenth hinging element 6127, may pivotally couple elongated slider 611 and coupling slab 612, by extending through coaxial horizontal bores 6117, 6117′ in slider 611 and through coaxial proximal through hole 6122 in coupling slab 612, axial groove 6116 in slider 611 adapted, sized, and configured to partially accommodate arcuate proximal end 6120 of coupling slab 612, according to some embodiments.

In an exemplary implementation as further illustrated in FIGS. 11-12B; swiveling end effector 600 may further include, according to some embodiments. a dorsal coupling member 3032, coupled to and extending distally from shaft distal end 3031, more specifically, sleeve 303, according to some embodiments. Dorsal coupling member 3032 may include: a distal portion 3008, defining dorsal aperture 3034, dorsal aperture 3034 being coaxial with aperture 6051 defined in dorsal hinge member 605 which may extend proximally from split shelf 6027 of second jaw 602; and proximal portion 3007, defining rectangular window 3035, rectangular window's 3035 narrow aspect sized and configured to accommodate tabs 6142, 6142′ of respective bracket slabs 614, 614′, wherein proximal portion 6100 of camming arm 613 may be sandwiched between tabs 6142, 6142′, according to some embodiments.

Additionally illustrated in FIG. 11 is ventral coupling member 3033, according to some embodiments, the ventral coupling member 3033 coupled to and having a distal portion 3006 (FIG. 12A) extending distally from distal end 3031 of shaft 300, distal portion 3006 defining ventral aperture 3005 which may be coaxial with aperture 6020 of horizontal partial beveled gear 6021 of partially circular extension 6002 of second jaw 602, according to some embodiments.

Third hinging element 3036′ (identical in function to third hinging element 3036, see e.g., FIG. 4A), according to some embodiments, pivotally couples dorsal coupling member 3032, via aperture 3034 therein, to dorsal hinge member, via aperture 6051 therein, and to split shelf 6027 of second jaw 602. Then fourth hinging element 3037′, may pivotally couple ventral coupling member 3033 and basal surface 6005 of partially circular extension 6002 of second jaw 602, by extending through ventral aperture 3005 of ventral coupling member 3033, and coaxial aperture 6020 of the partially circular extension 6002, according to some embodiments.

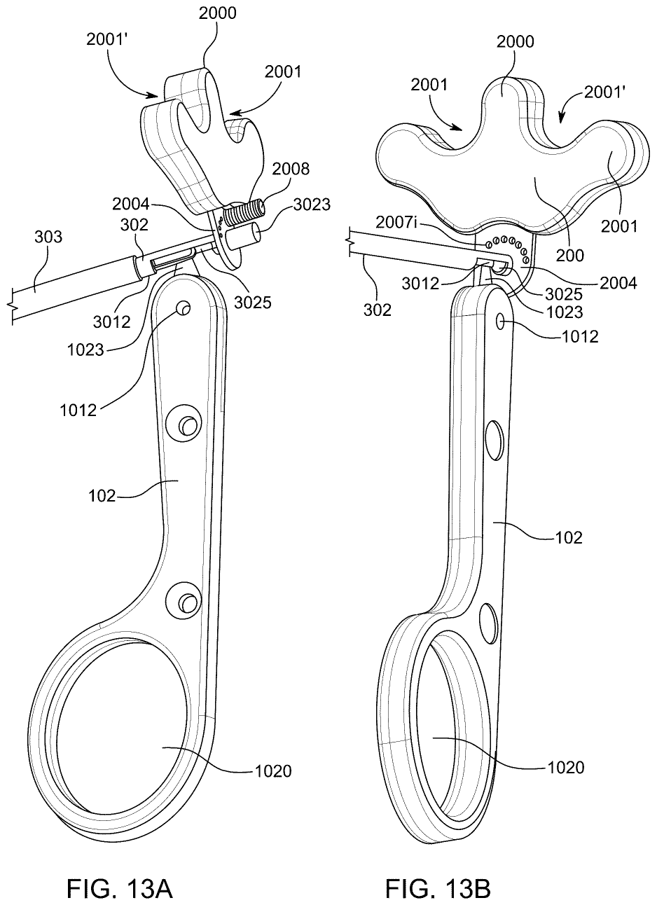

Turning now to FIGS. 13A-14B, where, as further illustrated, elongated cannula 302 of surgical instrument 60 may further include a ventral opening 3025 disposed toward proximal end 3023 of elongated cannula 302, according to some embodiments. In an exemplary implementation, stationary handle 101 may further include a head portion 1000 having a distal 1013 end, a proximal end 1002, and a body portion 1014, wherein head portion defines a sleeve bore 1015 coaxial with longitudinal axis of shaft 300 (see e.g., FIG. 1), according to some embodiments. Sleeve bore 1015 has a proximal end 1006 and a distal end coinciding with distal end 1013 of head portion 1000, according to some embodiments. Sleeve bore 1015 may be adapted, sized, and configured to accommodate a portion of swiveling member 200. A cavity 1005 in head portion 1000 may be adapted, sized, and configured to accommodate a biasing element 2008 and an indexing bearing 2009. Additionally head portion 1000 further defines: a hinge aperture 1050 configured to receive a sixth hinging element 105; slot 1004 extending through basal surface of head portion 1000 and through hinge aperture 1050 to sleeve bore 1015; and a partially circular dorsal slot 1008, extending to a cavity 1005, according to some embodiments.

As illustrated in FIGS. 13A-14B, articulating handle 102 may be configured to be partially accommodated in slot 1004 extending through basal surface of head portion 1000 of stationary handle 101, according to some embodiments, Articulating handle 102 may further defines an apical protrusion 1023, operable to allow pivotal coupling of the articulating handle 102 to proximal end 3102 of push-rod 3101 (see e.g., FIG. 13A). Apical protrusion 1023 may be hingedly coupled to stationary handle 101 via sixth hinging element 105, according to some embodiments. As further illustrated in FIG. 14B, rod assembly 301, further defines an axial bore 3105, rod assembly 301 terminating at rod assembly distal end 3010, the rod assembly distal end 3010 sized and configured to accommodate and engage various proximal ends (e.g., 7100, see e.g., FIG. 19) of other sub-assemblies coupled thereto, according to some embodiments.

Here too, swiveling member 200 may include: body portion 2000 defining right depression 2001 and left depression 2001′; flat neck portion 2004 coupled to body portion 2000, flat neck portion 2004 defining a partially circular surface, coupled to proximal end 3023 of elongated cannula 302, flat neck portion 2004 sized and configured to be accommodated within partially circular dorsal slot 1008 of head portion 1000 of stationary handle 101, wherein flat neck portion 2004 further defines an arcuate array of indexing holes or indexing depressions 2007i, according to some embodiments.

As further illustrated in FIGS. 14A, 14B, cavity 1005 may further include the biasing element 2008, operable to bias the bearing 2009 to engage one of the indexing holes or indexing depressions 2007i defined in the partially circular surface of the flat neck portion 2004 of swiveling member 200, according to some embodiments.

Another exemplary implementation of the surgical instrument is illustrated in FIGS. 16A-23B, according to some embodiments. Some components of this embodiment may be similar or identical in structure and/or functions to components of other embodiments, and will not be described again herein. As illustrated, for example in FIG. 16A-16D, surgical instrument 70, according to some embodiments, includes a shaft 700 comprising a sleeve 703, having a proximal end (not shown) coupled to stationary handle 101. A flaring 7034 at sleeve distal end 7001 may be hingedly coupled to end effector 800, wherein: a ventral opening 7037 may be defined toward sleeve distal end 7001, ventral opening 7037 configured to accommodate a portion of an end effector actuating assembly, according to some embodiments. A dorsal hinging plateau 7032 and a ventral hinging plateau 7033 are defined at sleeve distal end 7001, according to some embodiments. As further illustrated in FIG. 16D, elongated cannula 302 having distal portion 711, may be nested within and coaxial with sleeve 703, according to some embodiments. The proximal end 3023 of cannula 302 (FIG. 13A) may be operably coupled to swiveling member 200, and cannula distal end 7111 defines a partial vertical partial spur gear 7021 at its distal end 7111 (see e.g., FIG. 18A); and rod assembly 301, nested within elongated cannula 302 having distal portion 711, according to some embodiments. Rod assembly 301 has a proximal end 3100 (FIG. 14A) operably coupled to articulating handle 102, and a distal end 3010 coupled to an end effector actuating assembly (see e.g., FIG. 18B), according to some embodiments, which will be discussed further herein.

Turning now to FIG. 16C, second jaw 802 has a distal end 8025 and a proximal end 8028, the proximal end 8028 including a partially circular extension 8001 extending proximally from proximal end portions 8101, 8101′ of second jaw 802, according to some embodiments. Partially circular extension 8001 has a basal surface 8003, an apical surface 8002, and a horizontal partial spur gear 8021, extending from partially circular extension 8001, horizontal partial spur gear 8021 defining a coaxial aperture 8020 extending through partially circular extension 8001, wherein horizontal partial spur gear 8021 may be configured to engage vertical partial spur gear 7021 at cannula distal end 7111, according to some embodiments. Apically flaring open portions 8026, 8026′ form a shelf 8027 defining an opening 8005 sized and configured to accommodate a portion of first jaw 601, opening 8005 defined between proximal end portions 8101, 8101′, sized and configured to provide yaw swivel freedom of about ±2.09 rad., each of apically flaring open portions 8026, 8026′ also having pair of coaxial horizontal bores 8024, 8024′ defined therewithin, according to some embodiments. A shelf portion 80270 extends proximally from shelf 8027, defines an aperture 8029 therein, aperture 8029 being coaxial with aperture 8020 defined by partially circular extension 8001, wherein aperture 8029, and aperture 8020 are configured to receive third hinging element (swiveling pin) 3036″, according to some embodiments. Third hinging element 3036″ may also be configured to pivotally couple dorsal hinge plateau 7032, shelf 8027 of second jaw 802, coaxial aperture 8020 extending through circular extension 8001 of second jaw 802, and the ventral hinge plateau 7033, and slidably coupling portion of camming member 813, according to some embodiments.

Furthermore, and as illustrated, for example in FIG. 16B, first jaw 601 may include, according to some embodiments, a head portion 6010 having distal end 6015 (see e.g., FIG. 16B); rear section 6012 (see e.g., FIG. 12A) extending proximally from head portion 6010, rear section 6012 sized and configured to be partially accommodated within opening 8005 defined by apically flaring open portions 8026, 8026′, wherein rear section 6012 may be comprised of two lobes 6003, 6003′ (FIG. 12C), each lobe 6003, 6003′ defining a through hole 6004, 6004′, sized and configured to pivotally couple to apically flaring open portion 8026, 8026′ of second jaw 802, according to some embodiments.

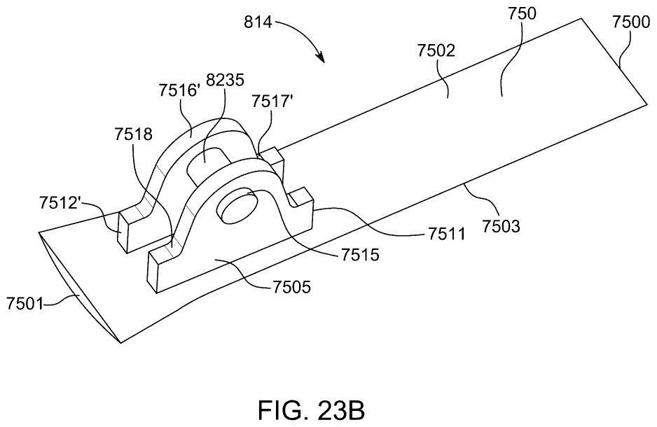

Turning now to FIGS. 16D-23B, there is illustrated yet another exemplary implementation of an end effector actuating assembly, according to some embodiments, which may include: a fulcrum member 814 (see e.g., FIG. 19) coupled to ventral opening 7037 (see e.g., FIG. 23A) in sleeve 703; and a camming member (also referred to as “camming element”) 813, having a proximal end 8130 and a frusto-spherical distal end 8131 coupled to circular depressions 6009, 6009′ defined in respective lobes 6003, 6003′ (FIG. 12C), the camming member 813 pivotally coupled to fulcrum member 814, as will be discussed further herein, according to some embodiments. A shuttle assembly 710 having a proximal end 7102 may be coupled to rod assembly distal end 3010, shuttle assembly 710 slidably coupled to camming member 813, wherein translation of shuttle assembly 710 distally within elongated cannula 302, may be configured to cause frusto-spherical distal end 8131 of camming element 813 to close first jaw 601, and wherein translation of shuttle assembly 710 proximally within elongated cannula 302 may be configured to cause frusto-spherical distal end 8131 of camming member 813 to open first jaw 601, according to some embodiments.

As illustrated for example in FIGS. 19, 23A, and 23B, fulcrum member 814 may include: a base plate 750 having proximal end 7500 and distal end 7501, with flat apical surface 7502 and arcuate basal surface 7503, arcuate basal surface 7503 having a curvature configured to complement a curvature of sleeve 703 adjacent to ventral opening 7037, according to some embodiments. Also, base plate 750 defines a pair of slits 7505, 7505′ (of which slit 7505 is shown in FIG. 16B), each slit 7505, 7505′ configured to receive a portion of a fulcrum bracket 751, 751′; and the pair of fulcrum brackets 751, 751′ may be separated by a predetermined gap D₈₂₃₅ (see e.g., FIG. 19), according to some embodiments. Each fulcrum bracket 751, 751′ has a proximal end 7511, 7511′, a distal end 7512, 7512′, a basal extension 7510, 7510′, and a partially circular portion 7516, 7516′ (see e.g., FIG. 23B) extending dorsally from proximal shelf 7517, 7517′ and distal shelf 7518, 7518′. Each fulcrum bracket 751, 751′ also defines a respective aperture 7515, 7515′, configured to receive fulcrum hinge 8235 (FIG. 23B), wherein each of basal extensions 7510, 7510′ may be configured to be accommodated and engaged within a corresponding one of slits 7505, 7505′ defined in base plate 750, according to some embodiments.

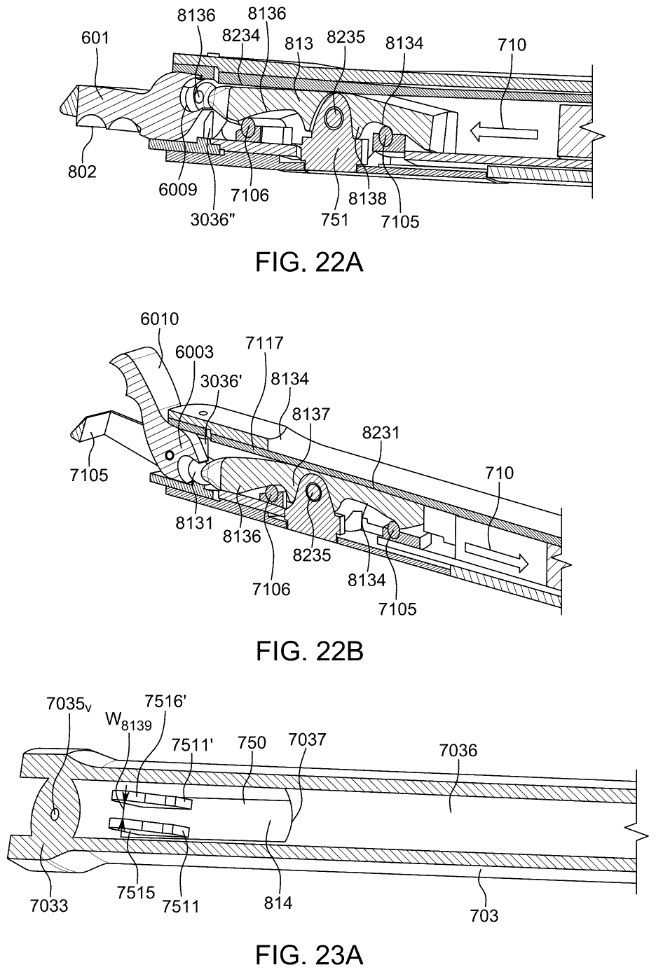

Furthermore, camming member 813 is illustrated for example, in FIG. 19, and 21 as having proximal end 8130, proximal portion 8231 having upper surface 8232, mid-section 8238, distal portion 8233 having upper surface 8234, and frusto-spherical distal end 8131, wherein: frusto-spherical distal end 8131 further defines bore 8132 having rounded rectangle cross section, configured to accommodate third hinging element 3036″ (see e.g., FIG. 22A), according to some embodiments. Proximal portion 8231 further defines arcuate basal surface 8134, where distal portion 8233 further defines arcuate basal surface 8136 that may be symmetric to arcuate basal surface 8134 defined in proximal portion 8231 of camming member 813, according to some embodiments.

As further illustrated, according to some embodiments, mid-section 8238 defines: partially circular extension 8139 defining co-axial bore 8135; arcuate rims 8230 (8230′) terminating in proximal edges 8138, 8138′ and distal edges 8137, 8137′ formed on either side of partially circular extension 8139, arcuate rims 8230, 8230′ having curvatures complimentary to curvatures defined by partially circular portions 7516, 7516′ of fulcrum members 751, 751′, wherein partially circular extension 8139 has a thickness W₈₁₃₉ (see e.g., FIG. 21) sized to be accommodated in gap D₈₂₃₅ predefined between pair of fulcrum members 751, 751′ (see FIG. 19), and wherein co-axial bore 8135 may be configured to accommodate fulcrum hinge 8235, according to some embodiments.

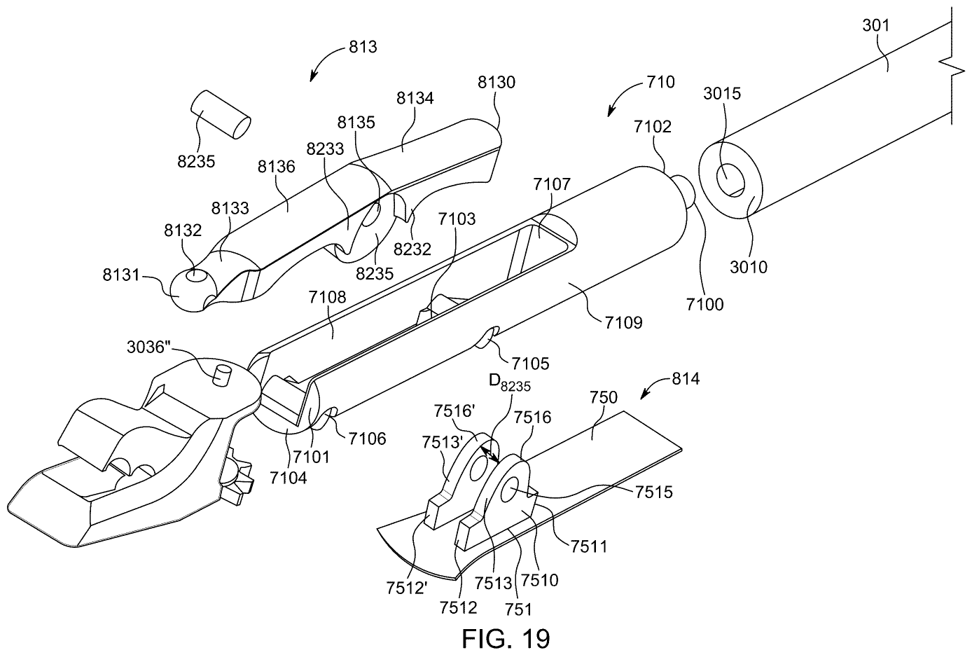

Turning now to FIG. 20, there is illustrated shuttle assembly 710, according to some embodiments, comprising an elongated cylindrical body 7109 defining a longitudinal axis XL₇₁₀ and having a proximal end 7102 and a distal end 7107. A proximal coupler 7100 extends proximally from shuttle assembly proximal end 7102, according to some embodiments. A pair of partially cylindrical tines 7200, 7200′ extends distally from distal end 7107 of elongated cylindrical body 7109 and terminates distally with common distal end 7101, according to some embodiments. Shuttle assembly 710 may further include a proximal beveled rib 7103 coupling partially cylindrical tines 7200, 7200′, whereby proximal beveled rib 7103 may be disposed between a pair of co-axial proximal apertures 7205, 7205′ defined in respective tines 7200, 7200′ and proximal beveled rib 7103 may be sized and configured to partially accommodate proximal bearing roller 7105, spanning gap W₇₂₀₀ (see e.g., FIG. 20) between partially cylindrical tines 7200, 7200′, according to some embodiments. A distal beveled rib 7104 couples partially cylindrical tines 7200, 7200′, distal beveled rib 7104 disposed between a pair of co-axial distal apertures 7204, 7204′ defined in respective tines 7200, 7200′, wherein distal beveled rib 7104 may be sized and configured to partially accommodate distal bearing roller 7106, spanning gap W₇₂₀₀ between partially cylindrical tines 7200, 7200′, according to some embodiments. Further, and as illustrated in FIGS. 22A, 22B, proximal bearing roller 7105 may be configured to abut arcuate basal surface 8134 of proximal portion 8231 of camming member 813, and wherein distal bearing roller 7106 may be configured to abut arcuate basal surface 8136 of distal portion 8233 of camming member 813, according to some embodiments.

Additionally, according to some embodiments, arcuate basal surface 8134 of proximal portion 8231 and arcuate basal surface 8136 of distal portion 8233 define respective apogees 81340, 81360 configured such that: when distal bearing roller 7106 may be beneath apogee 81360 (e.g., following proximal translation of shuttle assembly 710, see e.g., FIG. 22B), proximal bearing roller 7105 abuts arcuate basal surface 8134 of proximal portion 8231, causing camming member 813 to pivot about fulcrum member 814 and causing frusto-spherical distal end 8131 of camming member 813 to slide basally, and consequently causing first jaw 601 to open independently of yaw swivel angle, according to some embodiments. Conversely,, according to some embodiments, when proximal bearing roller 7105 may be beneath apogee 81340 (e.g., following distal translation of shuttle assembly 710, see e.g., FIG. 22A), distal bearing roller 7106 abuts arcuate basal surface 8136 of distal portion 8233, causing camming member 813 to pivot about fulcrum member 814 and to frusto-spherical distal end 8131 of camming member 813 to extend apically, and consequently first jaw 601 to close independently of yaw swivel angle, according to some embodiments.

As further illustrated in FIG. 22A, and 22B, upper surface 8232 of camming member proximal portion 8231 and upper surface 8234 of camming member distal portion 8233 are angled relative to each other, such that when actuated by translating shuttle assembly 710 distally (see e.g., FIG. 22A), upper surface 8234 of distal portion 8233 may be parallel with longitudinal axis XL₇₁₀, while upper surface 8232 of proximal portion 8231 may be angled relative to longitudinal axis XL₇₁₀, according to some embodiments. Conversely, when actuated by translating shuttle assembly 710 proximally (see e.g., FIG. 22B), upper surface 8232 of proximal portion 8231 may be parallel with longitudinal axis XL₇₁₀, while upper surface 8234 of distal portion 8233 may be angled relative to longitudinal axis XL₇₁₀, according to some embodiments.

Likewise, the axial distance L₈₂₃₅ (see e.g., FIG. 20) between the proximal beveled rib 7103, and the distal beveled rib 7104, may be sized to allow shuttle assembly 710 to translate about fulcrum brackets 751, 751′, while the distance D₇₁₀₇ between proximal beveled rib 7103 and distal end 7107 of elongated cylindrical body 7109, may be sized to partially accommodate proximal end 8130 of camming member 813, when shuttle assembly 710 may be translated distally (see e.g., FIG. 22A), according to some embodiments.

In certain exemplary implementations, the surgical instruments disclosed incorporate an upbite, or a downbite; the term “upbite” referring to an arrangement where the instrument removes material at a position above its longitudinal axis and the term “downbite” refers to an arrangement where the instrument removes material at a position below its longitudinal axis, according to some embodiments. For example, an upbite punch may be one where the cutting action takes place at a position angled upwardly relative to the remainder of the device, in other words, jaws that are angled upwardly relative to the remainder of the device for example about 5.0 mm. to 7.0 mm, and in particular relative to the position at which the instrument may be held, such as relative to handles that are opened or closed to effect opening or closing of the jaws, according to some embodiments.

Furthermore, the shaft can be configured to be curved, in other words, while sleeve 303 may be made of rigid materials, for example, stainless steel ph-17-4, 420, 465, or other super alloys, elongated cannula 302, and rod assembly 301 are made of a resilient material, allowing for purpose-specific curvature, according to some embodiments. For example, and in an exemplary implementation, the shaft may be upswept at a predetermined curvature, the shaft adapted and sized to follow the contour of the condyle, keeping a lower surface of the first jaw in parallel to the tibial plateau.

It will be appreciated by persons skilled in the art that a number of features of embodiments of the surgical instrument discussed herein may be provided in alternative embodiments. For example, any of the embodiments discussed herein may have an articulating handle which may be proximal to a stationary handle such as, for example, that shown in FIG. 7A or, alternatively, any of the embodiments discussed herein may be provided with an articulating handle which may be distal to a stationary handle such as, for example, that shown in FIG. 14A. Additionally, any of the embodiments discussed herein may be provided with a swiveling end effector as described herein with reference to any of FIGS. 3, 10, 15A-D, or 16A. Further, any of the embodiments discussed herein may include an end effector actuating assembly wherein the second jaw may be provided with a horizontal partial gear and a cannula may be provided with a vertical partial gear, wherein the gears may be selected from beveled gears, spur gears, or face gears. Yet further, any of the embodiments discussed herein may include an end effector actuating assembly including a camming element (for example, as discussed with reference to the embodiment of FIG. 4B), or a camming arm (for example, as discussed with reference to the embodiment of any of FIG. 12A-B or 12C), or a camming element (for example, as discussed with reference to FIG. 19) wherein the first jaw includes a lever having a depression corresponding to the distal end of the camming element/arm.

Additionally, it will be appreciated by persons skilled in the art that, instead of the end effector being configured to swivel in a horizontal plane, when the surgical instrument is viewed with the actuating handle and stationary handle being positioned below the shaft, optionally, components of the surgical instrument may be configured such that the end effector may be configured to swivel in a vertical plane, as will be understood by persons skilled in the art.

According to some embodiments, the surgical instrument may optionally be actuated by a robot instead of being handheld. Optionally, the surgical instrument may include an attachment to a robotic actuator, instead of actuating handle, according to some embodiments.

According to some embodiments, an end effector may be removable from the surgical instrument and may be replaced by another end effector. For example, any of the end effectors shown and described herein may be replaced by another of the end effectors shown and described herein. According to some embodiments, an end effector may be removed by withdrawing the third and fourth hinging elements (for example, such as those shown in FIG. 4A), removing the end effector 400, inserting a different end effector, and repositioning the third and fourth hinging elements relative to the components of the surgical instrument. With regard to the embodiment of FIG. 4A, this may include repositioning the third and fourth hinging elements relative to the dorsal coupling member 3032, the dorsal hinge member 405, the second jaw 402, and the ventral coupling member 3033, while ensuring that the ball joint 4030 of the camming element 403 is positioned within the depression 4019 in the first jaw lever 4006 (for example, as shown in FIGS. 3 and 4B), according to some embodiments. Similar repositioning of components may be performed for other embodiments discussed herein. There may be provided, therefore, a kit having a surgical instrument including a first end effector and at least one additional end effector. The end effectors may be any one of the end effectors described and shown herein, and additional end effectors having additional structures and functions, which may similarly replace an end effector in the surgical instrument, may be provided. Various embodiments and aspects of the present invention as delineated hereinabove and as claimed in the claims section below find experimental support in the following example.

Reference is now made to the following example, which together with the above descriptions illustrate some embodiments of the invention in a non-limiting fashion.

### Example I: Bite Force Measurement

A surgical instrument having a swiveling end effector such as, for example, shown and described herein, was attached to a measuring device at different swivel angles such as, 0 degrees, −45 degrees, 45 degrees, 90 degrees, and −90 degrees, as shown in the Table. Force was applied at a proximal portion of the surgical instrument such as, for example, at handle(s) of the surgical instrument, as discussed herein, using a spring load device. For each row of the Table, there is shown the measured bite force at the distal end of the surgical instrument, i.e., at the swiveling end effector, which resulted from a same force applied at the proximal portion of the surgical instrument. For example:

In the row showing 0.5 kg of bite force, the same amount of force (i.e., 1.0 kg) was applied at the surgical instrument proximal portion, to achieve the resulting bite force of 0.5 kg, regardless of the swivel angle of the end effector.

In the row showing 0.75 kg of bite force, for the same amount of force (i.e., 1.5 kg) was applied at the surgical instrument proximal portion, to achieve the resulting bite force of 0.75 kg, regardless of the swivel angle of the end effector.

In the row showing 1 kg of bite force, for the same amount of force (i.e., 2.0 kg) was applied at the surgical instrument proximal portion, to achieve the resulting bite force of 1 kg, regardless of the swivel angle of the end effector.

In the row showing 1.25 kg of bite force, for the same amount of force (i.e., 2.5 kg) was applied at the surgical instrument proximal portion, to achieve the resulting bite force of 1.25 kg, regardless of the swivel angle of the end effector.

In the row showing 1.5 kg of bite force, for the same amount of force (i.e., 3.0 kg) was applied at the surgical instrument proximal portion, to achieve the resulting bite force of 1.5 kg, regardless of the swivel angle of the end effector.

In the row showing 1.75 kg of bite force, for the same amount of force (i.e., 3.5 kg) was applied at the surgical instrument proximal portion, to achieve the resulting bite force of 1.75 kg, regardless of the swivel angle of the end effector.

In the row showing 2 kg of bite force, for the same amount of force (i.e., 4.0 kg) was applied at the surgical instrument proximal portion, to achieve the resulting bite force of 2 kg, regardless of the swivel angle of the end effector.

In the row showing 2.25 kg of bite force, for the same amount of force (i.e., 4.5 kg) was applied at the surgical instrument proximal portion, to achieve the resulting bite force of 2.25 kg, regardless of the swivel angle of the end effector.

As seen in the Table, the biting force was measured at different yaw angles (0°; +45°; and ±90°) relative to the shaft longitudinal axis. The force on the load cell was measured from 1 Kg to 4.5 Kg at 0.5 Kg increments. The measuring device has a 2 to 1 force ratio, according to some embodiments, so each measurement was divided by 2 as reported and shown in the Table.

### Instruments

Load Cell—Rinstrum R320

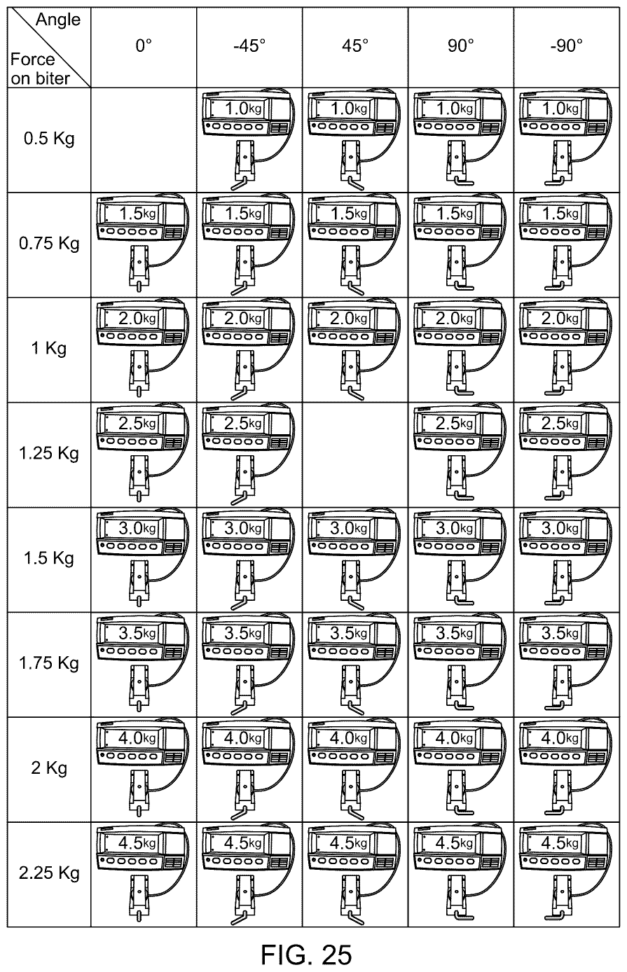

The table of FIG. 25 summarizes the results, as discussed herein.

The instrument (a punch, or “biter”) may indeed be operable to deliver the same torque independent of a yaw swivel angle.

While in the foregoing specification the surgical instrument having a swiveling end effector configured to deliver the same load at any yaw angle relative to a longitudinal axis of the surgical instrument provided herein have been described in relation to certain exemplary implementations, and many details are set forth for purpose of illustration, it will be apparent to those skilled in the art that the alignment methods disclosed herein, implementable using the systems disclosed herein, are not to be limited to implementations discussed herein, and that certain of the details described in this specification and as are more fully delineated in the following claims can be varied considerably without departing from the basic principles disclosed herein.

It is expected that during the life of a patent maturing from this application many relevant surgical instruments having end effectors will be developed and the scope of the term end effector is intended to include all such new technologies a priori.

As used herein the term “about” refers to ±10%.

The terms “comprises”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

As used herein, the term “treating” includes abrogating, substantially inhibiting, slowing or reversing the progression of a condition, substantially ameliorating clinical or aesthetical symptoms of a condition or substantially preventing the appearance of clinical or aesthetical symptoms of a condition.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

It is the intent of the Applicant(s) that all publications, patents and patent applications referred to in this specification are to be incorporated in their entirety by reference into the specification, as if each individual publication, patent or patent application was specifically and individually noted when referenced that it is to be incorporated herein by reference. In addition, citation or identification of any reference in this application shall not be construed as an admission that such reference is available as prior art to the present invention. To the extent that section headings are used, they should not be construed as necessarily limiting. In addition, any priority document(s) of this application is/are hereby incorporated herein by reference in its/their entirety.

1. A surgical instrument comprising:
a swiveling end effector at a distal portion of said surgical instrument, said swiveling end effector having a first jaw and a second jaw, said first jaw movable between an open position and a closed position relative to said second jaw, wherein said first jaw is actuatable by application of a force at a proximal portion of said surgical instrument and wherein said surgical instrument is configured to deliver a torque at said first jaw when moving between the open position and the closed position; and
a swiveling actuator coupled to said swiveling end effector and operative to swivel said swiveling end effector about a swivel axis;
wherein said surgical instrument is operable to deliver a substantially same torque at said first jaw when moving between the open position and the closed position, regardless of a swivel position of the swiveling end effector relative to said swivel axis.

2. The surgical instrument of claim 1, wherein when in said closed position said first jaw at least partially abuts said second jaw.

3. The surgical instrument of claim 1, wherein said surgical instrument includes a shaft extending from a proximal portion of said surgical instrument to said end effector at a distal portion of said surgical instrument, said shaft having a diameter of less than 4.5 mm, and wherein said surgical instrument is configured to generate torque on said first jaw of from about 150 N·mm to about 600 N·mm.

4. The surgical instrument of claim 1, wherein the swiveling end effector is one of a punch, a grasper, a suture passer, bypass scissors, and an anvil scissors.

5. A kit including:
the surgical instrument of claim 1; and
at least one additional swiveling end effector, wherein said swiveling end effector is replaceable with said additional swiveling end effector.

6. The surgical instrument of claim 1, wherein said surgical instrument has a longitudinal axis, and wherein said swiveling end effector is configured to swivel about ±120 degrees relative to said longitudinal axis, wherein said surgical instrument is operable to deliver a substantially same torque at said first jaw when moving between the open position and the closed position, at any swivel position of the swiveling end effector relative to said swivel axis selected from −120 degrees to +120 degrees.

7-8. (canceled)

9. The surgical instrument of claim 1, wherein said surgical instrument includes an end effector actuating assembly having a joint at which said first jaw is configured to hinge, wherein said swivel axis extends through said joint.

10. The surgical instrument of claim 1, wherein said surgical instrument includes an end effector actuating assembly having a joint at which said first jaw is configured to hinge, wherein said joint is a ball joint.

11. The surgical instrument of claim 1, wherein said end effector includes a biting tool configured to at least one of bite a meniscus in a knee and cut through a meniscus.

12. The surgical instrument of claim 1, wherein the surgical instrument is operable to deliver sufficient torque at said first jaw to move between the open position and the closed position.

13-16. (canceled)

17. An arthroscopic surgical instrument according to claim 1, wherein said first jaw is manually actuatable to move between the open position and the closed position relative to said second jaw.

18-19. (canceled)

20. The surgical instrument of claim 1, wherein said surgical instrument has a longitudinal axis;
wherein said surgical tool has a length of 5.5mm;
wherein said shaft has an outer diameter of 4.2 mm; and
wherein said surgical instrument is configured transmit a force applied at a proximal portion of said surgical instrument to generate torque of about 525 N-mm at a distal portion of said end effector, regardless of a swivel angle of said end effector relative to said longitudinal axis.

21. The surgical instrument of claim 1, wherein said swiveling end effector is configured to swivel both clockwise and counterclockwise about the swivel axis.

22-23. (canceled)

24. The surgical instrument according to claim 1, wherein said surgical instrument includes a proximal portion having an end effector actuator configured to receive a force and wherein said surgical instrument includes a distal portion including said end effector, wherein said end effector actuator is configured to transmit a force applied thereat and to convert the transmitted force to said torque to be applied to said first jaw to move said first jaw from said open position to said closed position.

25. The surgical instrument according to claim 1, wherein said surgical instrument includes a rotatable shaft having a distal end including a first gear, wherein said second jaw includes a proximal portion having a second gear, and wherein said first gear is configured to engage with said second gear whereby rotation of said shaft causes said first gear to turn said second gear thereby swiveling said swiveling end effector.

26. A method of using a surgical instrument having a longitudinal axis and a swiveling end effector including first and second jaws, said method including:
a. rotating a swiveling actuator about the longitudinal axis, wherein said rotation of said swiveling actuator is converted to swiveling of the swiveling end effector to a first swivel position at which the end effector is at a first selected angle relative to a longitudinal axis of the surgical instrument; and
b. applying torque in a first direction to a portion of the end effector to move the first jaw relative to the second jaw, from an open position to a closed position, wherein the amount of torque required to move the first jaw from the open position to the closed position is not dependent on a swivel position of the swiveling end effector;
wherein action (b) may be performed before action (a).

27. The method according to claim 26, further including applying torque in a second direction to the portion of the end effector to move the first jaw relative to the second jaw, from the closed position to the open position, wherein the second direction is opposite to the first direction.

28. A method according to claim 26, wherein the surgical instrument includes a shaft and wherein said rotating includes rotating the swiveling actuator a selected radial distance relative to the shaft to move the end effector from a first swivel orientation to a second swivel orientation.

29. A method according to claim 28, wherein said applying torque and said rotating a swiveling actuator may be performed with a same hand.

30. A method according to claim 26, wherein said surgical instrument includes a shaft having a longitudinal axis, and wherein said swiveling includes rotating the end effector about a swiveling axis, wherein said swiveling axis extends through the shaft longitudinal axis.

31. A method according to claim 26, further including:
c. swiveling the end effector to a second swivel position at which the end effector is at a second selected angle relative to the longitudinal axis of the surgical instrument.

32. A method according to claim 26, wherein the surgical instrument includes proximal and distal handles, and wherein said applying torque includes one of:
moving the proximal handle relative to the distal handle; and
moving the distal handle relative to the proximal handle.

33. (canceled)

34. A method according to claim 26, wherein the surgical instrument includes an end effector actuator having an axially displaceable first portion and wherein the first jaw includes a second portion configured to mate with the first portion, and wherein said applying torque includes axially displacing the first portion to apply torque to the second portion to move the first jaw relative to the second jaw.

35. A method according to claim 34, wherein one of:
said axially displaceable first portion includes a portion having at least a partially circular or spherical or cylindrical configuration, and wherein the second portion includes a depression or recess sized and shaped to correspond to the first portion; and
said second portion includes a portion having at least a partially circular or spherical or cylindrical configuration, and wherein said axially displaceable first portion includes a depression or recess sized and shaped to correspond to the second portion.

36-38. (canceled)

39. A surgical instrument comprising:
a swiveling end effector having a first jaw and a second jaw, the first jaw movable between an open position and a closed position partially abutting the second jaw, the surgical instrument operable to deliver a substantially same predetermined torque for closing the first jaw that is independent of a yaw swivel angle; and
a swiveling actuator coupled to said swiveling end effector and operative to swivel said swiveling end effector about a swivel axis.

40. The surgical instrument of claim 1, wherein said surgical instrument has a longitudinal axis; and
wherein said swiveling actuator is rotatable, whereby radial rotation of said swiveling actuator about the longitudinal axis is converted to swiveling of said swiveling end effector about said swivel axis, wherein said swivel axis is perpendicular to said longitudinal axis.
